SEQUENCE LISTING (24 sequences)

SEQ ID NO: 1 - protein, 117 aa, Artificial Sequence
  Synthetic Construct
QIQLVQSGPELKKPGETVKISCKASDYTFTDYSMHWVKQAPGKGLKWMGWINTETGEPTYADDFKGRFVFSLDTSVSTAYLQISSLKAEDTAVYYCARNKGYNLAYWGQGTLVTVSS

SEQ ID NO: 2 - DNA, 351 bp, Artificial Sequence
  Synthetic Construct
cagatccagttggtgcagtctggacctgagctgaagaagcctggagagacagtcaagatctcctgcaaggcttctgattataccttcacagactattcaatgcactgggtgaagcaggctccaggaaagggtttaaagtggatgggctggataaacactgagactggtgagccaacatatgcagatgacttcaagggacggtttgcctttcctttggaaacctctgccagcactgcctatttgcagatcaacaacctcaaaaatgaggacacggctacatatttctgtgctagaaatcatggttacaacctggcttactggggccaagggactctggtcactgtctctgca

SEQ ID NO: 3 - protein, 107 aa, Artificial Sequence
  Synthetic Construct
DIVMTQSQKFMSTSVGDRVSLTCKSSQNVRTAVTWYQQKPGQSPKALIYLASNRHTGVPDRFTGSGSGTDFTLTISNVQSEDLADYFCLQHWNYPLTFGGGTKLEIK

SEQ ID NO: 4 - DNA, 321 bp, Artificial Sequence
  Synthetic Construct
gacattgtgatgacccagtctcaaaaattcatgtccacatcagtaggagacagggtcagcctcacctgcaagtccagtcagaatgttcgtactgctgtaacctggtatcaacagaaaccagggcagtctcctaaagcactgatttacttggcatccaaccggcacactggagtccctgatcgcttcacaggcagtggatctgggacagattttactctcaccattagcaatgtgcaatctgaagacctggcagattatttctgtctgcaacattggaattatcctctcacgttcggaggggggaccaagctggaaataaaa

SEQ ID NO: 5 - protein, 117 aa, Artificial Sequence
  Synthetic Construct
EVQLQQSVAELVRPGASVKLSCTASGFNIKNTYMHWVKQRPEQGLEWIGRIDPANGNTKYAPKFQGKATITADTSSNTALQLSSLTSEDTAIYYCARWSTVVPMDYWGQGTSVTVSS

SEQ ID NO: 6 - DNA, 353 bp, Artificial Sequence
  Synthetic Construct
gaggttcagctgcagcagtctgtggcagagcttgtgaggccaggggcctcagtcaagttgtcctgcacagcttctggcttcaacattaaaaacacctatatgcactgggtgaagcagaggcctgaacagggcctggagtggattggaaggattgatcctgcgaatggtaatactaaatatgccccgaagttccagggcaaggccactataactgcagacacatcctccaacacagcctacctgcagctcagcagcctgacatctgaggacactgccatctattactgtgctagatggagacggtagtacctatggactactggggtcaaggaacctcagtcaccgtctcctca

SEQ ID NO: 7 - protein, 107 aa, Artificial Sequence
  Synthetic Construct
DIQMTQSPASQSASLGESVTITCLASQTIGTWLAWYQQKPGKSPQLLIYAATSLADGVPSRFSGSGSGTKFSFKISSLQAEDFVSYYCQQLYSTPYTFGGGTKLEIK

SEQ ID NO: 8 - DNA, 321 bp, Artificial Sequence
  Synthetic Construct
gacattcagatgacccagtctcctgcctcccagtctgcatctctgggagaaagtgtcaccatcacatgcctggcaagtcagaccattggtacatggttagcatggtatcagcagaaaccagggaaatctcctcagctcctgatttatgctgcaaccagcttggcagatggggtcccatcaaggttcagtggtagtggatctggcacaaaattttctttcaagatcagcagcctacaggctgaagattttgtaagttattactgtcaacaactttacagtactccgtacacgttcggaggggggaccaagctggaaataaaa

SEQ ID NO: 9 - protein, 119 aa, Artificial Sequence
  Synthetic Construct
QVQLQQPGTELVKPGASVKLSCKASGYTFTSYWMHWVKQRPGQGLEWIGNINPSNGGTNYNEKFKSKATLTVDKSSSTAYMQLSSLTSEDSAVYYCAREGTSWDRFDYWGQGTTLTVSS

SEQ ID NO: 10 - DNA, 357 bp, Artificial Sequence
  Synthetic Construct
caggtccaactgcagcagcctgggactgaactggtgaagcctggggcttcagtgaagctgtcctgcaaggcttctggctacaccttcaccagctactggatgcactgggtgaagcagaggcctggacaaggccttgagtggattggaaatattaatcctagcaatggtggtaccaactacaatgagaagttcaagagcaaggccacactgactgtagacaaatcctccagcacagcctacatgcagctcagcagcctgacatctgaggactctgcggtctattattgtgcaagagagggaactagctgggaccgctttgactactggggccaaggcaccactctcacagtctcctca

SEQ ID NO: 11 - protein, 110 aa, Artificial Sequence
  Synthetic Construct
DIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPYFGGGTKLEIK

SEQ ID NO: 12 - DNA, 333 bp, Artificial Sequence
  Synthetic Construct
gacattgtgctgacacagtctcctgcttccttagctgtatctctggggcagagggccaccatctcatgcagggccagccaaagtgtcagtacatctagctatagttatatgcactggtaccaacagaaaccaggacagccacccaaactcctcatcaagtatgcatccaacctagaatctggggtccctgccaggttcagtggcagtgggtctgggacagacttcaccctcaacatccatcctgtggaggaggaggatactgcaacatattactgtcagcacagttgggagattccgtacacgttcggaggggggaccaagctggaaataaaa

SEQ ID NO: 13 - protein, 119 aa, Artificial Sequence
  Synthetic Construct
EVKLVESGGGLVKPGGSLKLSCAASGFTFSSYAMSWVRQTPEKRLEWVASISSGGSTYYPDSVKGRFTISRDNARNILYLQMSSLRSEDTAMYYCARGRGYYAYYFDYWGQGTTLTVSS

SEQ ID NO: 14 - DNA, 357 bp, Artificial Sequence
  Synthetic Construct
gaagtgaagctggtggagtctgggggaggcttagtgaagcctggagggtccctgaaactctcctgtgcagcctctggattcactttcagtagctatgccatgtcttgggttcgccagactccagagaagaggctggagtgggtcgcatccattagtagtggtggtagcacctactatccagacagtgtgaagggccgattcaccatctccagagataatgccaggaacatcctgtacctgcaaatgagcagtctgaggtctgaggacacggccatgtattactgtgcaagaggcaggggttactacgcgtactactttgactactggggccaaggcaccactctcacagtctcctca

SEQ ID NO: 15 - protein, 107 aa, Artificial Sequence
  Synthetic Construct
ETTVTQSPASLSVATGEKVTIRCITSTDIDDDMNWYQQKPGEPPKLLISEGNTLRPGVPSRFSSSGYGTDFVFTIENTLSEDVADYYCLQSDNMPFTFGSGTKLEIK

SEQ ID NO: 16 - DNA, 321 bp, Artificial Sequence
  Synthetic Construct
gaaacaactgtgacccagtctccagcatccctgtccgtggctacaggagaaaaagtcactatcagatgcataaccagcactgatattgatgatgatatgaactggtaccagcagaagccaggggaacctcctaagctccttatttcagaaggcaatactcttcgtcctggagtcccatcccgattctccagcagtggctatggcacagattttgtttttacaattgaaaacacgctctcagaagatgttgcagattactactgtttgcaaagtgataacatgccattcacgttcggctcggggacaaagttggaaataaaa

SEQ ID NO: 17 - protein, 999 aa, Homo sapiens
MADKVRRQRPRRRVCWALVAVLLADLLALSDTLAVMSVDLGSESMKVAIVKPGVPMEIVLNKESRRKTPVIVTLKENERFFGDSAASMAIKNPKATLRYFQHLLGKQADNPHVALYQARFPEHELTFDPQRQTVHFQISSQLQFSPEEVLGMVLNYSRSLAEDFAEQPIKDAVITVPVFFNQAERRAVLQAARMAGLKVLQLINDNTATALSYGVFRRKDINTTAQNIMFYDMGSGSTVCTIVTYQMVKTKEAGMQPQLQIRGVGFDRTLGGLEMELRLRERLAGLFNEQRKGQRAKDVRENPRAMAKLLREANRLKTVLSANADHMAQIEGLMDDVDFKAKVTRVEFEELCADLFERVPGPVQQALQSAEMSLDEIEQVILVGGATRVPRVQEVLLKAVGKEELGKNINADEAAAMGAVYQAAALSKAFKVKPFVVRDAVVYPILVEFTREVEEEPGIHSLKHNKRVLFSRMGPYPQRKVITFNRYSHDFNFHINYGDLGFLGPEDLRVFGSQNLTTVKLKGVGDSFKKYPDYESKGIKAHFNLDESGVLSLDRVESVFETLVEDSAEEESTLTKLGNTISSLFGGGTTPDAKENGTDTVQEEEESPAEGSKDEPGEQVELKEEAEAPVEDGSQPPPPEPKGDATPEGEKATEKENGDKSEAQKPSEKAEAGPEGVAPAPEGEKKQKPARKRRMVEEIGVELVVLDLPDLPEDKLAQSVQKLQDLTLRDLEKQEREKAANSLEAFIFETQDKLYQPEYQEVSTEEQREEISGKLSAASTWLEDEGVGATTVMLKEKLAELRKLCQGLFFRVEERKKWPERLSALDNLLNHSSMFLKGARLIPEMDQIFTEVEMTTLEKVINETWAWKNATLAEQAKLPATEKPVLLSKDIEAKMMALDREVQYLLNKAKFTKPRPRPKDKNGTRAEPPLNASASDQGEKVIPPAGQTEDAEPISEPEKVETGSEPGDTEPLELGGPGAEPEQKEQSTGQKRPLKNDEL

SEQ ID NO: 18 - protein, 10 aa, Artificial Sequence
  Synthetic Construct
LQDLTLRDLE

SEQ ID NO: 19 - protein, 117 aa, Artificial Sequence
  Synthetic Construct
QVQLVQSGSELKKPGASVKVSCKASGYTFTDYSMHWVRQAPGQGLEWMGWINTETGEPTYADDFKGRFVFSLDTSVSTAYLQISSLKAEDTAVYYCARNKGYNLAYWGQGTLVTVSS

SEQ ID NO: 20 - protein, 107 aa, Artificial Sequence
  Synthetic Construct
DIQMTQSPFSLSASVGDRVTITCKSSQNVRTAVTWYQQKPGKAPKLLIYLASNRHTGVPSRFSGSGSGTDFTLTISSLQPEDFATYFCLQHWKYPLTFGGGTKLEIK

SEQ ID NO: 21 - protein, 19 aa, Artificial Sequence
  Synthetic Construct
MAWVWTLLFLMAAAQSIQA

SEQ ID NO: 22 - DNA, 57 bp, Artificial Sequence
  Synthetic Construct
atggcttgggtgtggaccttgctattcctgatggcggctgcccaaagtatccaagca

SEQ ID NO: 23 - protein, 24 aa, Artificial Sequence
  Synthetic Construct
MGIKMEFQTQVFVFVLLWLSGVDG

SEQ ID NO: 24 - DNA, 72 bp, Artificial Sequence
  Synthetic Construct
atgggcatcaagatggagtttcagacccaggtctttgtattcgtgttgctctggttgtctggtgttgatgga

(19) United States
(12) Patent Application Publication
(10) Pub. No.: US 2010/0135991 A1
(43) Pub. Date: Jun. 3, 2010

(54) ANTIBODIES RECOGNIZING OXYGEN-REGULATED PROTEIN 150 EXPRESSED ON CANCER CELLS AND METHODS OF USING SAME

(76) Inventors: Chiu-Chen Huang, Taipei (TW); Shih-Yao Lin, Taipei (TW); Leewen Lin, Taipei (TW)

Correspondence Address: MORRISON & FOERSTER LLP, 755 PAGE MILL RD, PALO ALTO, CA, 94304-1018, US

(21) Appl. No.: 12/620,331

(22) Filed: Nov. 17, 2009

Related U.S. Application Data
(60) Provisional application No. 61/115,436, filed on Nov. 17, 2008.
(60) Provisional application No. 61/249,201, filed on Oct. 6, 2009.

(51) Int. Cl. A61K 39/395; C07K 16/18; A61P 35/04; C07H 21/04; C12N 15/63; C12N 5/07; C07K 16/42; A61K 38/00; A61K 38/08; A61K 38/16; G01N 33/53

(52) U.S. Cl. 424/131.1; 530/387.9; 530/387.3; 530/391.7; 536/23.53; 435/320.1; 435/326; 424/139.1; 530/387.2; 514/2; 514/15; 514/12; 424/178.1; 435/7.21; 435/7.23

(57) ABSTRACT

The present invention provides novel antibodies that specifically bind to an ORP150 polypeptide expressed of the cell surface of plasmacytoma, multiple myeloma, colorectal cancer cells, gastric cancer cells, or esophageal cancer cells. These antibodies can be used in a variety of diagnostic and therapeutic methods.

### CROSS-REFERENCE TO RELATED APPLICATIONS

This application claims the priority benefit of U.S. provisional applications U.S. Ser. No. 61/115,436, filed Nov. 17, 2008, and U.S. Ser. No. 61/249,201, filed Oct. 6, 2009, all of which are incorporated herein by reference in their entirety.

### FIELD OF THE INVENTION

The present invention relates to novel antibodies that recognize an ORP150 polypeptide expressed on the cell surface of plasmacytoma cells, multiple myeloma cells, colorectal cancer cells, gastric cancer cells, and/or esophageal cancer cells. These antibodies have the property of inducing cell death (e.g., apoptosis) in plasmacytoma cells, multiple myeloma cells, and gastric cancer cells in the absence of cytotoxin conjugation and immune effector function. These antibodies can be used to induce complement-dependent cytotoxicity or antibody-dependent cell-mediated cytotoxicity in plasmacytoma cells, multiple myeloma cells, colorectal, gastric or esophageal cancer cells after binding to the cell surface of the cells. These antibodies are useful as diagnostic and therapeutic agents.

### BACKGROUND OF THE INVENTION

Multiple myeloma is a systemic malignancy of plasma cells that is highly treatable but rarely curable. The estimated frequency of multiple myeloma is 5 to 7 new cases per 100,000 persons per year. Accordingly, in the United States, 19,920 new cases are expected to be diagnosed in 2008. There were more than 56,000 Americans living with multiple myeloma in 2005.

Multiple myeloma is often referred to by the particular type of immunoglobulin or light chain (kappa or lambda type) produced by the cancerous plasma cell. The frequency of the various immunoglobulin types of multiple myeloma parallels the normal serum concentrations of the immunoglobulins. The most common multiple myeloma types are IgG and IgA. IgG multiple myeloma accounts for about 60% to 70% of all cases of multiple myeloma, and IgA accounts for about 20% of cases. Few cases of IgD and IgE multiple myeloma have been reported. Although a high level of monoclonal protein in the blood is a hallmark of multiple myeloma disease, about 15% to 20% of patients with multiple myeloma produce incomplete immunoglobulins, containing only the light chain portion of the immunoglobulin. In a rare form of multiple myeloma called nonsecretory multiple myeloma, plasma cells do not produce M protein or light chains. This affects about 1% of multiple myeloma patients.

Proper staging of myeloma helps in determining prognosis and developing a treatment plan. The Durie-Salmon system has been the most widely used myeloma staging system since 1975. In this system, the clinical stage of disease (stage I, II, or III) is based on several measurements, including levels of M protein, the number of bone lesions, hemoglobin values, and serum calcium levels. In the Durie-Salmon system, stages are further divided according to renal function as determined by serum creatinine levels (classified as A or B).

Colorectal cancer is cancer that develops in the colon (the longest part of the large intestine) and/or the rectum (the last several inches of the large intestine before the anus). Colorectal cancer is the third most commonly diagnosed cancer in both men and women in the United States. The American Cancer Society estimates that there will be 148,610 new cases of colorectal cancer diagnosed in the U.S. this year. One out of 18 people in this country will develop colorectal cancer in their lifetime. Colorectal cancer can be divided into stages 0 (least severe), I, II, III, and IV (most severe).

Due to the limitations of current treatments for multiple myeloma, colorectal cancer, gastric cancer, and esophageal cancer, there remains a significant interest in and need for additional or alternative therapies for treating, stabilizing, preventing, and/or delaying development of these cancers. Desirably, the therapies overcome the shortcomings of current chemotherapy, radiation, and cell transplantation treatments.

All references, publications, and patent applications disclosed herein are hereby incorporated by reference in their entirety.

### BRIEF SUMMARY OF THE INVENTION

The invention provides antibodies (and polypeptides derived from them) that specifically bind to an ORP150 polypeptide expressed on the cell surface of a plasmacytoma cell, multiple myeloma cell, colorectal cancer cell, gastric cancer cell, or esophageal cancer cell. In some embodiments, the antibody induces apoptosis of the plasmacytoma cell, the multiple myeloma cell, or gastric cancer cell after binding to the cell surface of the cells in the absence of cytotoxin conjugation and immune effector function. In some embodiments, the antibody induces complement-dependent cytotoxicity in the cell after binding to the cell surface of the cell. In some embodiments, the antibody induces antibody-dependent cell-mediated cytotoxicity in the cell after binding to the cell surface of the cell. In some embodiments, the antibody does not specifically bind one or more of the following cells (e.g., human cells): embryonic vein endothelial cells or peripheral blood cells, such as T-lymphocytes, B-lymphocytes, monocytes, neutrophils, platelets, or red blood cells. In some embodiments, the antibody does not specifically bind embryonic vein endothelial cells, T-lymphocytes, B-lymphocytes, monocytes, neutrophils, platelets, or red blood cells. In some embodiments, no detectable binding is observed between the antibody and any of the following cells (e.g., human cells): embryonic vein endothelial cells, T-lymphocytes, B-lymphocytes, monocytes, neutrophils, platelets, or red blood cells. In some embodiments, the antibody is conjugated to a cytotoxin. In some embodiments, the antibody is a monoclonal antibody. In some embodiments, the antibody is a murine, a human, a humanized, or a chimeric antibody. In some embodiments, the human, humanized or chimeric antibody is of IgG1, IgG2, IgG3, or IgG4 isotype. In some embodiments, the antibody is a bispecific antibody.

In some embodiments, the antibody described herein, upon binding to an epitope expressed on the cell surface of the plasmacytoma, multiple myeloma, colorectal cancer cell, gastric cancer cell, or esophageal cancer cell, reduces the number of cancer cells and/or inhibits growth or proliferation of the cancer cell. For example, the reduction in cell number or inhibition of cell growth in the presence the antibody is by at least any of about 10%, about 20%, about 30%, about 40%, about 50%, about 65%, about 75%, or greater as compared to cell number or cell growth in the absence of the antibody.

In some embodiments, the antibody described herein recognizes an epitope on an extracellular domain of an ORP150 polypeptide. In some embodiments, the antibody binds to an epitope within amino acid residues 723-732, 673-752, 701-800, or 673-800 of human ORP150 polypeptide (SEQ ID NO:17). In some embodiments, the antibody binds to a polypeptide comprising amino acids 723-732, 673-752, 701-800, or 673-800 of SEQ ID NO:17.

In some embodiments, the antibody described herein competes with antibody 5F4, 3B6.1, 6A4.28, or 9A6.2 for binding to an ORP150 polypeptide (such as binding to an epitope in an extracellular domain of the ORP150 polypeptide) present on the cell surface of the plasmacytoma, multiple myeloma, colorectal cancer cell, gastric cancer cell, or esophageal cancer cell. In some embodiments, the antibody described herein competes with an antibody comprising the heavy chain variable region of antibody 5F4 and/or the light chain variable region of 5F4 for binding to an ORP150 polypeptide (such as binding to an epitope in an extracellular domain of the ORP150 polypeptide) present on the cell surface of the plasmacytoma, multiple myeloma, colorectal cancer cell, gastric cancer cell, or esophageal cancer cell. In some embodiments, the antibody described herein competes with an antibody comprising the heavy chain variable region of antibody 3B6.1 and/or the light chain variable region of 3B6.1 for binding to an ORP150 polypeptide (such as binding to an epitope in an extracellular domain of the ORP150 polypeptide) present on the cell surface of the plasmacytoma, multiple myeloma, colorectal cancer cell, gastric cancer cell, or esophageal cancer cell. In some embodiments, the antibody described herein competes with an antibody comprising the heavy chain variable region of antibody 6A4.28 and/or the light chain variable region of 6A4.28 for binding to an ORP150 polypeptide (such as binding to an epitope in an extracellular domain of the ORP150 polypeptide) present on the cell surface of the plasmacytoma, multiple myeloma, colorectal cancer cell, gastric cancer cell, or esophageal cancer cell. In some embodiments, the antibody described herein competes with an antibody comprising the heavy chain variable region of antibody 9A6.2 and/or the light chain variable region of 9A6.2 for binding to an ORP150 polypeptide (such as binding to an epitope in an extracellular domain of the ORP150 polypeptide) present on the cell surface of the plasmacytoma, multiple myeloma, colorectal cancer cell, gastric cancer cell, or esophageal cancer cell.

In some embodiments, the antibody comprises a heavy chain variable region comprising the three CDRs of antibody 5F4, and/or a light chain variable region comprising the three CDRs of antibody 5F4. In some embodiments, the antibody comprises the heavy chain variable region of antibody 5F4 and/or the light chain variable region of antibody 5F4. In some embodiments, the antibody is a chimeric antibody or a humanized antibody of IgG1, IgG2, IgG3, or IgG4 isotype.

In some embodiments, the antibody comprises a heavy chain variable region comprising the three CDRs from SEQ ID NO:19, and/or a light chain variable region comprising the three CDRs from SEQ ID NO:20. In some embodiments, the antibody comprises a heavy chain variable region comprising the amino acid sequence of SEQ ID NO:19 and/or the light chain variable region comprising the amino acid sequence of SEQ ID NO:20. In some embodiments, the antibody is a chimeric antibody or a humanized antibody of IgG1, IgG2, IgG3, or IgG4 isotype.

In some embodiments, the antibody comprises a heavy chain variable region comprising the three CDRs of antibody 3B6.1, and/or a light chain variable region comprising the three CDRs of antibody 3B6.1. In some embodiments, the antibody comprises the heavy chain variable region of antibody 3B6.1 and/or the light chain variable region of antibody 3B6.1. In some embodiments, the antibody is a chimeric antibody or a humanized antibody of IgG1, IgG2, IgG3, or IgG4 isotype.

In some embodiments, the antibody comprises a heavy chain variable region comprising the three CDRs of antibody 6A4.28, and/or a light chain variable region comprising the three CDRs of antibody 6A4.28. In some embodiments, the antibody comprises the heavy chain variable region of antibody 6A4.28 and/or the light chain variable region of antibody 6A4.28. In some embodiments, the antibody is a chimeric antibody or a humanized antibody of IgG1, IgG2, IgG3, or IgG4 isotype.

In some embodiments, the antibody comprises a heavy chain variable region comprising the three CDRs of antibody 9A6.2, and/or a light chain variable region comprising the three CDRs of antibody 9A6.2. In some embodiments, the antibody comprises the heavy chain variable region of antibody 9A6.2 and/or the light chain variable region of antibody 9A6.2. In some embodiments, the antibody is a chimeric antibody or a humanized antibody of IgG1, IgG2, IgG3, or IgG4 isotype.

In another aspect, the invention features anti-idiotypic antibodies (and polypeptides derived from them) that specifically bind to a first antibody, wherein the first antibody specifically binds to an ORP150 polypeptide expressed on the cell surface of a plasmacytoma cell, multiple myeloma cell, colorectal cancer cell, gastric cancer cell, or esophageal cancer cell. In some embodiments, the anti-idiotypic antibody specifically binds antibody 5F4, 3B6.1, 6A4.28, or 9A6.2. In some embodiments, the anti-idiotypic antibody is a monoclonal antibody. In some embodiments, the anti-idiotypic antibody is a murine, a human, a humanized, or a chimeric antibody.

In another aspect, the present invention provides polypeptides comprising a heavy chain and/or light chain, or a fragment of an antibody described herein. The invention also provides polypeptides derived from any of the antibodies described herein, wherein the polypeptides specifically bind to an ORP150 polypeptide expressed on the cell surface of a plasmacytoma cell, multiple myeloma cell, colorectal cancer cell, gastric cancer cell, or esophageal cancer cell. In some embodiments, the fragment of an antibody specifically binds to an ORP150 polypeptide expressed on the cell surface of a plasmacytoma cell, multiple myeloma cell, colorectal cancer cell, gastric cancer cell, or esophageal cancer cell.

In another aspect, the present invention provides polynucleotides encoding any of the antibodies or polypeptides described herein. The invention also provides vectors (such as expression vectors) comprising any of the polynucleotides described herein. The invention also provides host cells comprising any of the polynucleotides or vectors described herein.

In another aspect, the present invention provides a composition comprising any of the antibodies or polypeptides described herein. In certain embodiments, the antibodies or the polypeptides are linked to an agent. In some embodiments, the agent is a therapeutic agent (e.g., a radioactive moiety, cytotoxin, or chemotherapeutic agent). In some embodiments, the agent is a label (e.g., an enzyme, fluorescent molecule, or biotin). In some embodiments, the composition includes an anti-idiotypic antibody of the invention and a polypeptide comprising an ORP150 polypeptide or fragment thereof (such as an extracellular domain) expressed on the cell surface of a plasmacytoma cell, multiple myeloma cell, colorectal cancer cell, gastric cancer cell, or esophageal cancer cell. In some embodiments, the polypeptide comprises amino acid residues 723-732, 673-752, 701-800, or 673-800 of SEQ ID NO:17.

The invention also provides a pharmaceutical composition comprising an effective amount of any of the antibodies or polypeptides described herein, or polynucleotides encoding the antibodies or polypeptides, and a pharmaceutically acceptable carrier. In some embodiments, the antibodies or the polypeptides are linked to a therapeutic agent. In some embodiments, the composition is formulated for administration by intraperitoneal, intravenous, subcutaneous, or intramuscular injections, or other forms of administration such as oral, mucosal, via inhalation, sublingually, etc.

In some embodiments, the composition comprises more than one antibody or polypeptide of the invention, or one antibody of the invention with one or more other anti-cancer antibodies or other anti-cancer agents. In some embodiments, the composition comprises one antibody or polypeptide of the invention.

In another aspect, the invention features a composition comprising a polypeptide comprising an ORP150 polypeptide or fragment thereof obtained from or expressed on the cell surface of a plasmacytoma cell, multiple myeloma cell, colorectal cancer cell, gastric cancer cell, or esophageal cancer cell. In some embodiments, the composition is a pharmaceutical composition (such as a vaccine composition) that includes (i) an effective amount of an ORP150 polypeptide or fragment thereof obtained from or expressed on the cell surface of a plasmacytoma cell, multiple myeloma cell, colorectal cancer cell, gastric cancer cell, or esophageal cancer cell and (ii) a pharmaceutically acceptable carrier. In some embodiments, the ORP150 polypeptide fragment comprises an extracellular domain or fragment thereof from an ORP150 polypeptide expressed on the cell surface of a plasmacytoma cell, multiple myeloma cell, colorectal cancer cell, gastric cancer cell, or esophageal cancer cell. In some embodiments, the composition comprises a polypeptide comprising the amino acid residues 723-732, 673-752, 701-800, or 673-800 of SEQ ID NO:17. In some embodiments, the composition is formulated for administration by intraperitoneal, intravenous, subcutaneous, or intramuscular injections, or other forms of administration such as oral, mucosal, via inhalation, sublingually, etc. In some embodiments, the composition includes an adjuvant and/or a therapeutic agent.

In another aspect, the present invention provides methods for generating an antibody or a polypeptide described herein comprising culturing a host cell or progeny thereof under conditions that allow production of the antibody or polypeptide, wherein the host cell comprises an expression vector that encodes for the antibody or the polypeptide. In some embodiments, the method further comprises purifying the antibody or the polypeptide.

In another aspect, the invention provides an antibody or polypeptide generated by expressing a polynucleotide encoding the antibody or the polypeptide.

In another aspect, the invention provides methods of generating any of the antibodies or polypeptides described herein by expressing one or more polynucleotides encoding the antibody or polypeptide (which may be separately expressed as a single light or heavy chain, or both a light and a heavy chain are expressed from one vector) in a suitable cell, generally followed by recovering and/or isolating the antibody or polypeptides of interest.

In another aspect, the invention provides methods of generating any of the antibodies or polypeptides described herein by administering a polypeptide comprising an ORP150 polypeptide or fragment thereof (such as an extracellular domain) to an animal, and selecting an antibody or polypeptide produced by the animal that specifically binds to an ORP150 polypeptide expressed on the cell surface of a plasmacytoma cell, multiple myeloma cell, colorectal cancer cell, gastric cancer cell, or esophageal cancer cell. In some embodiments, the antibody or polypeptide is isolated. In some embodiments, part or all of a cell membrane comprising an ORP150 polypeptide or fragment thereof is administered to the animal. In some embodiments, a polypeptide comprising the amino acid residues 723-732, 673-752, 701-800, 673-800 of SEQ ID NO:17 is administered to the animal. In some embodiments, the ORP150 polypeptide fragment comprises an extracellular domain or fragment thereof from an ORP150 polypeptide expressed on the cell surface of a plasmacytoma cell, multiple myeloma cell, colorectal cancer cell, gastric cancer cell, or esophageal cancer cell. In some embodiments, the animal is a mammal (such as a non-human mammal). In some embodiments, antibody generated is further screened for its ability to induce cell death (e.g., through apoptosis) of a plasmacytoma cell, multiple myeloma cell, colorectal cancer cell, gastric cancer cell, and/or esophageal cancer cell after binding to the cell surface of the cells in the absence of cytotoxin conjugation and immune effector function.

In another aspect, the invention provides methods of generating any of the antibodies or polypeptides described herein by selecting antibodies or polypeptides that specifically binds to an ORP150 polypeptide expressed on the cell surface of a plasmacytoma cell, multiple myeloma cell, colorectal cancer cell, gastric cancer cell, or esophageal cancer cell from an antibody or polypeptide library (such as a phage display library). In some embodiments, antibody generated is further screened for its ability to induce cell death (e.g., through apoptosis) of a plasmacytoma cell, multiple myeloma cell, colorectal cancer cell, gastric cancer cell, and/or esophageal cancer cell after binding to the cell surface of the cells in the absence of cytotoxin conjugation and immune effector function.

In another aspect, the invention provides methods of generating an anti-idiotypic antibody by administering to an animal an antibody or fragment thereof that specifically binds to an ORP150 polypeptide expressed on the cell surface of a plasmacytoma cell, multiple myeloma cell, colorectal cancer cell, gastric cancer cell, or esophageal cancer cell. An anti-idiotypic antibody produced by the animal that specifically binds to the administered antibody or fragment thereof is selected. In some embodiments, the anti-idiotypic antibody is isolated. In some embodiments, the animal is a mammal (such as a non-human mammal).

In another aspect, the present invention provides methods for inducing apoptosis (e.g., in vitro) in a plasmacytoma cell, multiple myeloma cell, or gastric cancer cell expressing an ORP150 polypeptide on the cell surface, comprising contacting the plasmacytoma cell, multiple myeloma cell, or the gastric cancer cell with one or more antibodies or polypeptides described herein that bind to an ORP150 polypeptide expressed on the cell surface of a plasmacytoma cell, multiple myeloma cell, or gastric cancer cell. In some embodiments, the plasmacytoma cell, multiple myeloma cell, or gastric cancer cell is in an individual. In some embodiments, the one or more antibodies bind to the cell surface of a plasmacytoma, multiple myeloma cell or gastric cancer cell in the individual. In some embodiments, the antibody or polypeptide induces apoptosis of the plasmacytoma cell, the multiple myeloma cell, or gastric cancer after binding to the cell surface of the plasmacytoma cell or the multiple myeloma cell in the absence of cytotoxin conjugation and immune effector function. In some embodiments, the antibody or polypeptide induces complement-dependent cytotoxicity in the cell after binding to the cell surface of the cell. In some embodiments, the antibody or polypeptide induces antibody-dependent cell-mediated cytotoxicity in the cell after binding to the cell surface of the cell. In some embodiments, the antibody or polypeptide is conjugated to a cytotoxin (such as a cytotoxin that kills a plasmacytoma cell or multiple myeloma cell).

In another aspect, the present invention provides methods for treating plasmacytoma, multiple myeloma, colorectal cancer, gastric cancer or esophageal cancer in an individual comprising administering to the individual an effective amount of a composition comprising one or more antibodies or polypeptides described herein (such as (i) an antibody that specifically binds to an ORP150 polypeptide expressed on the cell surface of a plasmacytoma cell, multiple myeloma cell, colorectal cancer cell, gastric cancer cell or esophageal cancer cell or (ii) an anti-idiotypic antibody). In some embodiments, the one or more antibodies or polypeptides bind to the cell surface of a plasmacytoma, multiple myeloma, colorectal cancer cell, gastric cancer cell, or esophageal cancer cell in the individual. In some embodiments, the antibody or polypeptide induces apoptosis of the plasmacytoma cell, the multiple myeloma cell or gastric cancer cell after binding to the cell surface of the plasmacytoma cell, the multiple myeloma cell, or the gastric cancer cell in the absence of cytotoxin conjugation and immune effector function. In some embodiments, the antibody or polypeptide induces complement-dependent cytotoxicity in the cell after binding to the cell surface of the cell. In some embodiments, the antibody or polypeptide induces antibody-dependent cell-mediated cytotoxicity in the cell after binding to the cell surface of the cell. In some embodiments, the antibody or polypeptide is conjugated to a cytotoxin (such as a cytotoxin that kills a plasmacytoma cell, multiple myeloma cell, colorectal cancer cell, gastric cancer cell, or esophageal cancer cell).

In another aspect, the present invention provides methods for treating plasmacytoma, multiple myeloma, colorectal cancer, gastric cancer, or esophageal cancer in an individual comprising administering to the individual (i) one or more antibodies or polypeptides described herein and (ii) another anti-cancer agent, whereby the antibody (or polypeptide) and the anti-cancer agent in conjunction provide effective treatment of the plasmacytoma, multiple myeloma, colorectal cancer, gastric cancer or esophageal cancer in the individual. In some embodiments, the one or more antibodies or polypeptides bind to the cell surface of a plasmacytoma, multiple myeloma, colorectal cancer cell, gastric cancer cell, or esophageal cancer cell in the individual. In some embodiments, the anti-cancer agent is a chemotherapeutic agent. In some embodiments, the antibody or polypeptide induces apoptosis of the plasmacytoma cell, the multiple myeloma cell, or the gastric cancer cell after binding to the cell surface of the plasmacytoma cell, the multiple myeloma cell, or the gastric cancer cell in the absence of cytotoxin conjugation and immune effector function (e.g., in vitro). In some embodiments, the antibody or polypeptide induces complement-dependent cytotoxicity in the cell after binding to the cell surface of the cell. In some embodiments, the antibody or polypeptide induces antibody-dependent cell-mediated cytotoxicity in the cell after binding to the cell surface of the cell. In some embodiments, the antibody or polypeptide is conjugated to a cytotoxin (such as a cytotoxin that kills a plasmacytoma cell, multiple myeloma cell, colorectal cancer cell, gastric cancer cell, or esophageal cancer cell).

In another aspect, the present invention features methods for preventing or delaying the development of plasmacytoma, multiple myeloma, colorectal cancer, gastric cancer, or esophageal cancer in an individual having plasmacytoma, multiple myeloma, colorectal cancer, gastric cancer, or esophageal cancer or having an increased risk for plasmacytoma, multiple myeloma, colorectal cancer, gastric cancer, or esophageal cancer, comprising administering to the individual an effective amount of one or more antibodies or polypeptides described herein (such as (i) an antibody that specifically binds to an ORP150 polypeptide expressed on the cell surface of a plasmacytoma cell, multiple myeloma cell, colorectal cancer cell, gastric cancer cell, or esophageal cancer cell or (ii) an anti-idiotypic antibody). In some embodiments, the one or more antibodies or polypeptides bind to the cell surface of a plasmacytoma, multiple myeloma, colorectal cancer cell, gastric cancer cell, or esophageal cancer cell in the individual. In some embodiments, the antibody or polypeptide induces apoptosis of the plasmacytoma cell, the multiple myeloma cell, or gastric cancer cell after binding to the cell surface of the plasmacytoma cell, the multiple myeloma cell, or the gastric cancer cell in the absence of cytotoxin conjugation and immune effector function. In some embodiments, the antibody or polypeptide induces complement-dependent cytotoxicity in the cell after binding to the cell surface of the cell. In some embodiments, the antibody or polypeptide induces antibody-dependent cell-mediated cytotoxicity in the cell after binding to the cell surface of the cell. In some embodiments, the antibody or polypeptide is conjugated to a cytotoxin (such as a cytotoxin that kills a plasmacytoma cell, multiple myeloma cell, colorectal cancer cell, gastric cancer cell, or esophageal cancer cell).

In another aspect, the present invention provides methods for treating plasmacytoma, multiple myeloma, colorectal cancer, gastric cancer, or esophageal cancer in an individual having plasmacytoma, multiple myeloma, colorectal cancer, gastric cancer, or esophageal cancer comprising administering to the individual an effective amount of a polypeptide comprising an ORP150 polypeptide or fragment thereof obtained from or expressed on the cell surface of a plasmacytoma cell, multiple myeloma cell, colorectal cancer cell, gastric cancer cell, or esophageal cancer cell (such as a vaccine composition). In some embodiments, one or more antibodies that bind to the cell surface of a plasmacytoma, multiple myeloma, colorectal cancer cell, gastric cancer cell, or esophageal cancer cell in the individual are generated by the individual. In some embodiments, one or more of the antibodies that are generated reduce the number of cancer cells and/or inhibit cell growth or proliferation of tumor or cancer cells (e.g., plasmacytoma cells, multiple myeloma cells, colorectal cancer cells, gastric cancer cells, or esophageal cancer cells) that express an ORP150 polypeptide in the individual. In some embodiments, the ORP150 polypeptide fragment is an extracellular domain or fragment thereof from an ORP150 polypeptide expressed on the cell surface of a plasmacytoma cell, multiple myeloma cell, colorectal cancer cell, gastric cancer cell, or esophageal cancer cell. In some embodiments, a polypeptide comprising amino acid residues 723-732, 673-752, 701-800, or 673-800 of SEQ ID NO:17 is administered to the individual.

A method for preventing or delaying the development of plasmacytoma, multiple myeloma, colorectal cancer, gastric cancer, or esophageal cancer in an individual having plasmacytoma, multiple myeloma, colorectal cancer, gastric cancer, or esophageal cancer or having an increased risk for plasmacytoma, multiple myeloma, colorectal cancer, gastric cancer, or esophageal cancer, comprising administering to the individual an effective amount of a polypeptide comprising an ORP150 polypeptide or fragment thereof of obtained from or expressed on the cell surface of a plasmacytoma cell, multiple myeloma cell, colorectal cancer cell, gastric cancer cell, or esophageal cancer cell. In some embodiments, one or more antibodies that bind to the cell surface of a plasmacytoma, multiple myeloma, colorectal cancer cell, gastric cancer cell, or esophageal cancer cell in the individual are generated by the individual. In some embodiments, one or more of the antibodies that are generated reduce the number of cancer cells and/or inhibit cell growth or proliferation of tumor or cancer cells (e.g., plasmacytoma cells, multiple myeloma cells, colorectal cancer cells, gastric cancer cells, or esophageal cancer cells) that express an ORP150 polypeptide in the individual. In some embodiments, the ORP150 polypeptide fragment is an extracellular domain or fragment thereof from an ORP150 polypeptide expressed on the cell surface of a plasmacytoma cell, multiple myeloma cell, colorectal cancer cell, gastric cancer cell, or esophageal cancer cell. In some embodiments, a polypeptide comprising amino acid residues 723-732, 673-752, 701-800, or 673-800 of SEQ ID NO:17 is administered to the individual.

In another aspect, the present invention provides methods for detecting or diagnosing plasmacytoma, multiple myeloma, colorectal cancer, gastric cancer, or esophageal cancer; identifying an individual having plasmacytoma, multiple myeloma, colorectal cancer, gastric cancer, or esophageal cancer for treatment; or monitoring progression of plasmacytoma, multiple myeloma, colorectal cancer; gastric cancer, or esophageal cancer comprising contacting a sample with an antibody or a polypeptide described herein that binds to an ORP150 polypeptide expressed on the cell surface of a plasmacytoma cell, multiple myeloma cell, colorectal cancer cell, gastric cancer, or esophageal cancer; and detecting the presence or absence, or level of binding of the antibody or the polypeptide to the cell surface of a cell in the sample. The presence of binding between the antibody (or polypeptide) and the cell surface of a cell in the sample indicates that the sample may contain a cancer cell (such as a plasmacytoma cell, multiple myeloma cell, colorectal cancer cell, gastric cancer cell, or esophageal cancer cell), and/or the individual having cancer may be treated with an antibody or polypeptide described herein. The methods may further comprise a step of comparing the level of binding to a control.

In another aspect, the present invention provides methods for diagnosing plasmacytoma or multiple myeloma or an increased risk for plasmacytoma or multiple myeloma in an individual comprising contacting a bone marrow sample from the individual with of one or more antibodies or polypeptides described herein that bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of a bone marrow cell indicates that the individual has plasmacytoma or multiple myeloma or has an increased risk for plasmacytoma or multiple myeloma.

In another aspect, the present invention provides methods for diagnosing colorectal cancer or an increased risk for colorectal cancer in an individual comprising contacting a sample comprising a colon or rectum cell (such as a biopsy or other surgical sample) from the individual with of one or more antibodies or polypeptides described herein that bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of the colon or rectum cell indicates that the individual has colorectal cancer or has an increased risk for colorectal cancer.

In another aspect, the present invention provides methods for diagnosing gastric cancer or an increased risk for gastric cancer in an individual comprising contacting a sample comprising a gastric cell (such as a biopsy or other surgical sample) from the individual with of one or more antibodies or polypeptides described herein that bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of the gastric cell indicates that the individual has gastric cancer or has an increased risk for gastric cancer.

In another aspect, the present invention provides methods for diagnosing esophageal cancer or an increased risk for esophageal cancer in an individual comprising contacting a sample comprising a esophageal cell (such as a biopsy or other surgical sample) from the individual with of one or more antibodies or polypeptides described herein that bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of the esophageal cell indicates that the individual has esophageal cancer or has an increased risk for esophageal cancer.

In another aspect, the present invention provides methods for selecting a therapy for an individual having plasmacytoma or multiple myeloma comprising contacting a sample comprising a bone marrow sample from the individual with of one or more antibodies or polypeptides described herein that bind to an ORP150 polypeptide, whereby the ability of one or more of the antibodies or polypeptides to bind to the cell surface of a bone marrow cell indicates that the one or more antibodies or polypeptides are useful for treating the plasmacytoma or multiple myeloma in the individual.

In another aspect, the present invention provides methods for selecting a therapy for an individual having colorectal cancer in an individual comprising contacting a sample comprising a colon or rectum cell (such as a biopsy or other surgical sample) from the individual with of one or more antibodies or polypeptides described herein that bind to an ORP150 polypeptide, whereby the ability of one or more of the antibodies or polypeptides to bind to the cell surface of the colon or rectum cell indicates that the one or more antibodies or polypeptides are useful for treating the colorectal cancer in the individual.

In another aspect, the present invention provides methods for selecting a therapy for an individual having gastric cancer in an individual comprising contacting a sample comprising a gastric cell (such as a biopsy or other surgical sample) from the individual with of one or more antibodies or polypeptides described herein that bind to an ORP150 polypeptide, whereby the ability of one or more of the antibodies or polypeptides to bind to the cell surface of the gastric cell indicates that the one or more antibodies or polypeptides are useful for treating the gastric cancer in the individual.

In another aspect, the present invention provides methods for selecting a therapy for an individual having esophageal cancer in an individual comprising contacting a sample comprising a esophageal cell (such as a biopsy or other surgical sample) from the individual with of one or more antibodies or polypeptides described herein that bind to an ORP150 polypeptide, whereby the ability of one or more of the antibodies or polypeptides to bind to the cell surface of the esophageal cell indicates that the one or more antibodies or polypeptides are useful for treating the esophageal cancer in the individual.

In another aspect, the present invention provides kits for treating plasmacytoma, multiple myeloma, colorectal cancer, gastric cancer, or esophageal cancer in an individual comprising an antibody or a polypeptide described herein (such as (i) an antibody that specifically binds to an ORP150 polypeptide expressed on the cell surface of a plasmacytoma cell, multiple myeloma cell, colorectal cancer cell, gastric cancer cell, or esophageal cancer cell or (ii) an anti-idiotypic antibody). These kits may further comprise instructions for administering an effective amount of the antibody or the polypeptide to the individual for treating plasmacytoma, multiple myeloma, colorectal cancer, gastric cancer or esophageal cancer. In some embodiments, the antibody or polypeptide is in a pharmaceutical composition.

In another aspect, the invention provides kits that include any of the antibodies, polypeptides, or composition described herein. In some embodiments, the present invention provides kits for preventing or delaying the development of plasmacytoma, multiple myeloma, colorectal cancer, gastric cancer, or esophageal cancer in an individual comprising an antibody or a polypeptide described herein. These kits may further comprise instructions for administering an effective amount of the antibody or the polypeptide to the individual for preventing or delaying the development of plasmacytoma, multiple myeloma, colorectal cancer, gastric cancer, or esophageal cancer. In some embodiments, the antibody or polypeptide is in a pharmaceutical composition.

In another aspect, the present invention provides kits for treating plasmacytoma, multiple myeloma, colorectal cancer, gastric cancer, or esophageal cancer in an individual comprising a polypeptide comprising an ORP150 polypeptide or fragment thereof obtained from or expressed on the cell surface of a plasmacytoma cell, multiple myeloma cell, colorectal cancer cell, gastric cancer cell, or esophageal cancer cell. These kits may further comprise instructions for administering an effective amount of the polypeptide comprising an ORP150 polypeptide or fragment to an individual for treating plasmacytoma, multiple myeloma, colorectal cancer, gastric cancer, or esophageal cancer. In some embodiments, the ORP150 polypeptide fragment is an extracellular domain or fragment thereof from an ORP150 polypeptide expressed on the cell surface of a plasmacytoma cell, multiple myeloma cell, colorectal cancer cell, gastric cancer cell, or esophageal cancer cell. In some embodiments, the polypeptide comprises the amino acids 723-732, 673-752, or 701-800 of SEQ ID NO:17. In some embodiments, the polypeptide is in a pharmaceutical composition.

In another aspect, the present invention provides kits for preventing or delaying the development of plasmacytoma, multiple myeloma, colorectal cancer, gastric cancer, or esophageal cancer in an individual comprising a polypeptide comprising an ORP150 polypeptide or fragment thereof obtained from or expressed on the cell surface of a plasmacytoma cell, multiple myeloma cell, colorectal cancer cell, gastric cancer cell, or esophageal cancer cell. These kits may further comprise instructions for administering an effective amount of the ORP150 polypeptide or fragment to an individual for preventing or delaying the development of plasmacytoma, multiple myeloma, colorectal cancer, gastric cancer, or esophageal cancer. In some embodiments, the ORP150 polypeptide fragment is an extracellular domain or fragment thereof from an ORP150 polypeptide expressed on the cell surface of a plasmacytoma cell, multiple myeloma cell, colorectal cancer cell, gastric cancer cell, or esophageal cancer cell. In some embodiments, the polypeptide comprises the amino acids 723-732, 673-752, 701-800, or 673-800 of SEQ ID NO:17. In some embodiments, the polypeptide is in a pharmaceutical composition.

In another aspect, the present invention provides kits for detecting or diagnosing plasmacytoma, multiple myeloma, colorectal cancer, gastric cancer, or esophageal cancer; identifying an individual having plasmacytoma, multiple myeloma, colorectal cancer, gastric cancer, or esophageal cancer for treatment; or monitoring progression of plasmacytoma, multiple myeloma, colorectal cancer gastric cancer, or esophageal cancer; comprising an antibody or a polypeptide described herein that binds to an ORP150 polypeptide expressed on the cell surface of a plasmacytoma cell, multiple myeloma cell, colorectal cancer cell, gastric cancer cell, or esophageal cancer cell. In some embodiments, the kit includes one or more reagents for detecting binding of the antibody or the polypeptide to a cell in a sample. In some embodiments, the kit includes instructions for detecting or diagnosing plasmacytoma, multiple myeloma, colorectal cancer, gastric cancer, or esophageal cancer; identifying an individual having plasmacytoma, multiple myeloma, colorectal cancer, gastric cancer, or esophageal cancer for treatment; or monitoring progression of plasmacytoma, multiple myeloma, colorectal cancer, gastric cancer, or esophageal cancer.

In one aspect, the invention features an antibody or polypeptide (such as any of the antibodies or polypeptides described herein) for use as a medicament. In some embodiments, the invention features an antibody or polypeptide for use in a method of treating, preventing, or delaying the development of plasmacytoma, multiple myeloma, colorectal cancer, gastric cancer, or esophageal cancer in an individual. In some embodiments, the invention features the use of an antibody or polypeptide (such as any of the antibodies or polypeptides described herein) for the manufacture of a medicament, such as a medicament for treating, preventing, or delaying the development of plasmacytoma, multiple myeloma, colorectal cancer, gastric cancer, or esophageal cancer in an individual.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIG. 1 is a picture of a Western blot showing that antibody 5F4 recognizes anti-ORP150-immunoprecipitated protein.

FIGS. 2A and 2B are sequences of the murine 5F4 variable region. FIG. 2A shows the amino acid (SEQ ID NO:1) and nucleic acid (SEQ ID NO:2) sequence of the mature heavy chain. The amino acid (SEQ ID NO:21) and nucleic acid sequence (SEQ ID NO:22) of the heavy chain signal peptide are also shown in FIG. 2A. FIG. 2B shows the amino acid (SEQ ID NO:3) and nucleic acid (SEQ ID NO:4) sequence of the mature light chain. The amino acid (SEQ ID NO:23) and nucleic acid sequence (SEQ ID NO:24) of the light chain signal peptide are also shown FIG. 2B. Complementary determining regions are underlined.

FIGS. 3A and 3B are sequences of the murine 3B6.1 variable region. FIG. 3A is the amino acid (SEQ ID NO:5) and nucleic acid (SEQ ID NO:6) sequence of the mature heavy chain, and FIG. 2B is the amino acid (SEQ ID NO:7) and nucleic acid (SEQ ID NO:8) sequence of the mature light chain. Complementary determining regions are underlined.

FIGS. 4A and 4B are sequences of the murine 6A4.28 variable region. FIG. 4A is the amino acid (SEQ ID NO:9) and nucleic acid (SEQ ID NO:10) sequence of the mature heavy chain, and FIG. 4B is the amino acid (SEQ ID NO:11) and nucleic acid (SEQ ID NO:12) sequence of the mature light chain. Complementary determining regions are underlined.

FIGS. 5A and 5B are sequences of the murine 9A6.2 variable region. FIG. 5A is the amino acid (SEQ ID NO:13) and nucleic acid (SEQ ID NO:14) sequence of the mature heavy chain, and FIG. 5B is the amino acid (SEQ ID NO:15) and nucleic acid (SEQ ID NO:16) sequence of the mature light chain. Complementary determining regions are underlined.

FIG. 6 is a graph showing different isotypes of chimeric 5F4 antibodies (c5F4-hIgG1, c5F4-hIgG2, c5F4-hIgG3, and c5F4-hIgG4) induced apoptosis of human plasmacytoma cell line U266 in vitro. U266 cells were incubated with human Ig (a human Ig mixture, Scottish National Transfusion Service, Cat#F000685) or a chimeric 5F4 antibody at 1 ug/ml, 3 ug/ml, 10 ug/ml or 30 ug/ml in the presence of cross-linker (AffinityPure mouse anti-human IgG Fcγ fragment specific, Jackson ImmunoResearch, Cat#209-005-098) for 6 hours. At the end of the incubation, cells were stained with Yo-Pro-1 and the percentage of stained cells was measured using FACS analysis.

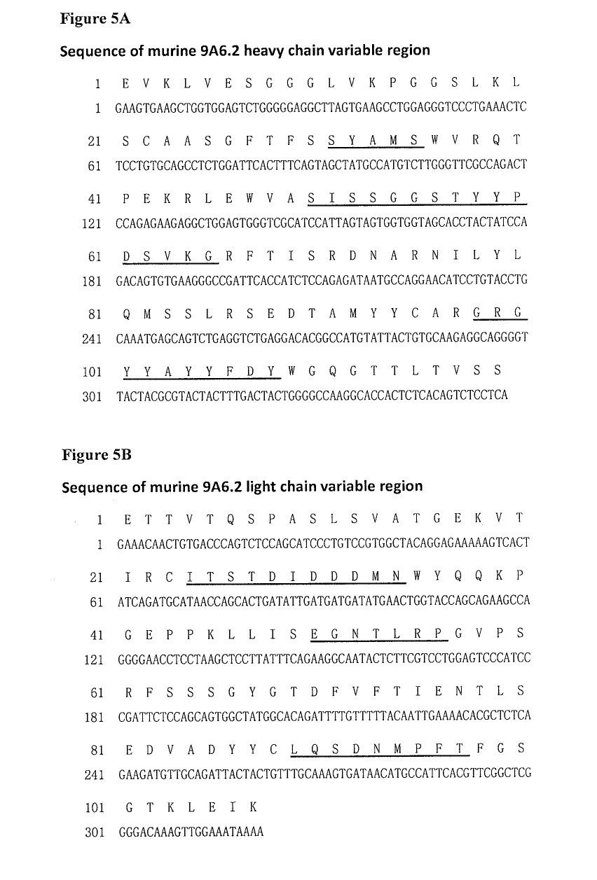

FIGS. 7A and 7B are graphs showing different isotypes of h5F4Ac.2/v17 induced comparable level of apoptosis of human plasmacytoma cell line NCI-H929 and U266 in vitro. NCI-H929 cells (FIG. 7A) or U266 (FIG. 7B) were incubated with human IgG3 or a IgG1 (h5F4Ac.2/v17_G1), IgG2 (h5F4Ac.2/v17_G2) or IgG3 (h5F4Ac.2/v17_G3) of humanized antibody h5F4Ac.2/v17 at 1 ug/ml, 3 ug/ml, 10 ug/ml or 30 ug/ml in the presence of cross-linker (AffinityPure mouse anti-human IgG Fcγ fragment specific, Jackson ImmunoResearch, Cat#209-005-098) for 6 hours. At the end of the incubation, cells were double-stained with Annexin V-FITC and PI and the percentage of stained cells was measured using FACS analysis.

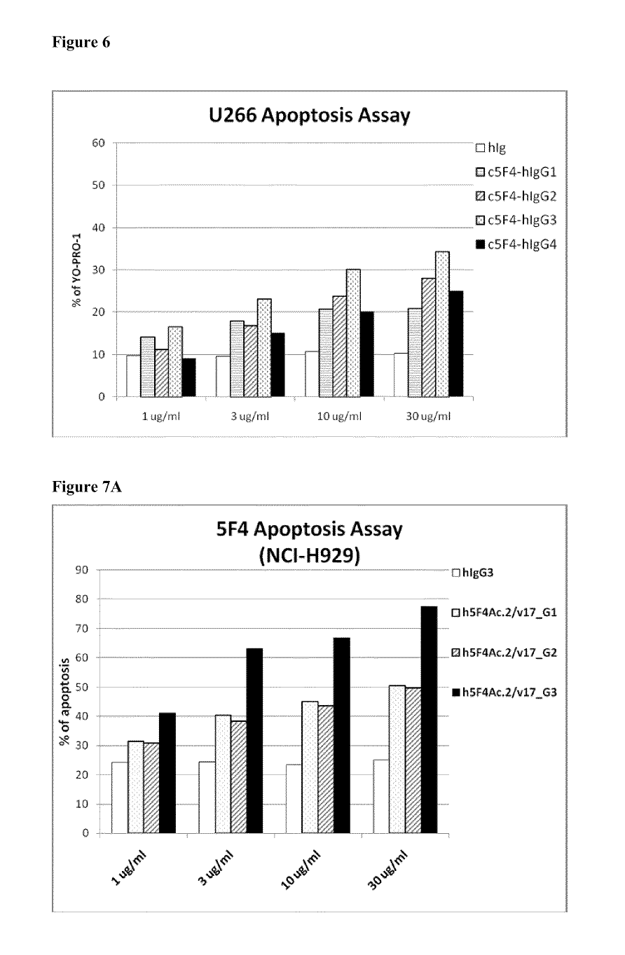

FIG. 8 is a graph showing suppression of multiple myeloma tumor growth in SCID mice by a humanized 5F4 antibody. Mice were implanted subcutaneously with L363 cells and treated intraperitoneally with humanized 5F4Ac.2/v17 or a control human IgG at 20 mg/kg three times weekly. Tumor volume was determined at day 5, 7, 9, 14, 16, 19, 21 and 23 after tumor implantation.

### DETAILED DESCRIPTION OF THE INVENTION

### Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively. The subunit structures and three-dimensional configurations of different classes of immunoglobulins are well known.

The antibody of the present invention is further intended to include bispecific, multispecific, single-chain, chimeric, and humanized molecules having affinity for a polypeptide conferred by at least one CDR region of the antibody. Antibodies of the present invention also include single domain antibodies which are either the variable domain of an antibody heavy chain or the variable domain of an antibody light chain. Holt et al., Trends Biotechnol. 21:484-490, 2003. Methods of making domain antibodies comprising either the variable domain of an antibody heavy chain or the variable domain of an antibody light chain, containing three of the six naturally occurring complementarity determining regions from an antibody, are also known in the art. See, e.g., Muyldermans, Rev. Mol. Biotechnol. 74:277-302, 2001.

As used herein, “monoclonal antibody” refers to an antibody of substantially homogeneous antibodies, i.e., the individual antibodies comprising the population are identical except for possible naturally-occurring mutations that may be present in minor amounts. Monoclonal antibodies are generally highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

As used herein, a “chimeric antibody” refers to an antibody having a variable region or part of a variable region from a first species and a constant region from a second species. An intact chimeric antibody comprises two copies of a chimeric light chain and two copies of a chimeric heavy chain. The production of chimeric antibodies is known in the art (Cabilly et al. (1984), Proc. Natl. Acad. Sci. USA, 81:3273-3277; Harlow and Lane (1988), Antibodies: a Laboratory Manual, Cold Spring Harbor Laboratory). Typically, in these chimeric antibodies, the variable region of both light and heavy chains mimics the variable regions of antibodies derived from one species of mammal, while the constant portions are homologous to the sequences in antibodies derived from another. One clear advantage to such chimeric forms is that, for example, the variable regions can conveniently be derived from presently known sources using readily available hybridomas or B-cells from non-human host organisms in combination with constant regions derived from, for example, human cell preparations. While the variable region has the advantage of ease of preparation, and the specificity is not affected by its source, the constant region being human is less likely to elicit an immune response from a human subject when the antibodies are injected than would the constant region from a non-human source. However, the definition is not limited to this particular example. In some embodiments, amino acid modifications are made in the variable and/or constant region.

An “isolated” antibody is one which has been identified and separated and/or recovered from a component of its natural environment.

As used herein, “substantially pure” refers to material which is at least 50% pure (i.e., free from contaminants), more desirably at least 90% pure, more desirably at least 95% pure, more desirably at least 98% pure, more desirably at least 99% pure.

As used herein, “humanized” antibodies refer to forms of non-human (e.g., murine) antibodies that are specific chimeric immunoglobulins, immunoglobulin chains, or fragments thereof (such as Fv, Fab, Fab′, F(ab′)₂, or other antigen-binding subsequences of antibodies) that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody.

As used herein, “human antibody” means an antibody having an amino acid sequence corresponding to that of an antibody produced by a human and/or has been made using any of the techniques for making human antibodies known in the art or disclosed herein. This definition of a human antibody includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B-lymphocytes that produce an antibody directed against a target antigen (such B-lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda Md.)); and (2) an approach based on crystallographic studies of antigen-antibody complexes (Al-lazikani et al (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination. A constant region of an antibody generally provides structural stability and other biological functions such as antibody chain association, secretion, transplacental mobility, and complement binding, but is not involved with binding to the antigen. The amino acid sequence and corresponding exon sequences in the genes of the constant region is dependent upon the species from which it is derived; however, variations in the amino acid sequence leading to allotypes is relatively limited for particular constant regions within a species. The variable region of each chain is joined to the constant region by a linking polypeptide sequence. The linkage sequence is coded by a “J” sequence in the light chain gene, and a combination of a “D” sequence and a “J” sequence in the heavy chain gene.

As used herein, “idiotope” and “idiotypic determinant” refer to an antigenic determinant or epitope in a primary antibody. In some embodiments, the idiotope is unique to the immunoglobulin product of a single clone of cells. In some embodiments, the idiotope is found in the variable region of the primary antibody. In some embodiments, the idiotope is a framework-associated or regulatory idiotope in the primary antibody.

An “anti-idiotypic antibody” refers to an antibody that specifically binds to an idiotope of a primary antibody. In some embodiments, the anti-idiotypic antibody specifically binds to one or more antibodies of the invention (such as an antibody that recognizes an ORP150 polypeptide expressed on the cell surface of plasmacytoma cells, multiple myeloma cells, and/or colorectal cancer cells).

As used herein, “antibody-dependent cell-mediated cytotoxicity” and “ADCC” refer to a cell-mediated reaction in which nonspecific cytotoxic cells that express Fc receptors (FcRs) (e.g., natural killer (NK) cells, neutrophils, or macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in a animal model such as that disclosed in Clynes et al., 1998, PNAS (USA), 95:652-656.

“Complement dependent cytotoxicity” and “CDC” refer to the lysing of a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santoro et al., J. Immunol. Methods, 202:163 (1996), may be performed.

The terms “polypeptide,” “oligopeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon an antibody, the polypeptides can occur as single chains or associated chains.

“Polynucleotide,” or “nucleic acid,” as used interchangeably herein, refer to polymers of nucleotides of any length, and include DNA and/or RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses, lyxoses, pyranose sugars, furanose sugars, sedoheptuloses, acyclic analogs, and abasic nucleoside analogs such as methyl ribosides. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO, or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl, or araldyl. Not all linkages in a polynucleotide need be identical. The preceding description applies to all polynucleotides referred to herein, including RNA and DNA.

As used herein, “vector” means a construct that is capable of delivering and desirably expressing one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

As used herein, an “effective dosage” or “effective amount” of drug, compound, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective dosage can be administered in one or more administrations. For purposes of this invention, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during, or after administration of the other treatment modality to the individual.

As used herein, “treatment” or “treating” is an approach for obtaining beneficial or desired results, including desirably clinical results. For purposes of this invention, beneficial or desired clinical results include, but are not limited to, one or more of the following: reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “individual” or a “subject” is a mammal, more desirably a human. Mammals also include, but are not limited to, farm animals, sport animals, pets (such as cats, dogs, or horses), primates, mice, and rats.

As use herein, the term “specifically recognizes” or “specifically binds” refers to measurable and reproducible interactions such as attraction or binding between a target and an antibody that is determinative of the presence of the target in the presence of a heterogeneous population of molecules including biological molecules. For example, an antibody that specifically or preferentially binds to an epitope is an antibody that binds this epitope with greater affinity, avidity, more readily, and/or with greater duration than it binds to other epitopes of the target or non-target epitopes. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of greater than or about 10³ M⁻¹ or about 10⁴ M⁻¹, sometimes about 10⁵ M⁻¹ or about 10⁶ M⁻¹, in other instances about 10⁶ M⁻¹ or about 10⁷ M⁻¹, about 10⁸ M⁻¹ to about 10⁹ M⁻¹, or about 10¹⁰ M⁻¹ to about 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, the terms “cancer,” “tumor,” “cancerous,” and “malignant” refer to or describe the physiological condition in mammals that is typically characterized by unregulated cell growth. Examples of cancer include plasmacytoma, multiple myeloma, and colorectal cancer.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

Reference to “about” a value or parameter herein includes (and describes) embodiments that are directed to that value or parameter per se. For example, description referring to “about X” includes description of “X.”

It is understood that aspect and variations of the invention described herein include “consisting” and/or “consisting essentially of” aspects and variations.

### Overview of ORP150 Polypeptides

The invention provides antibodies that recognize an oxygen-regulated protein 150 (also denoted an ORP150 polypeptide or a hypoxia up-regulated 1 precursor, HYOU-1) and methods of use. Originally discovered in hypoxic rat astrocytes, ORP150 polypeptides belong to the heat shock protein family. Heat shock proteins are some of the most abundant intracellular proteins. They normally function as molecular chaperones, assisting with folding of nascent polypeptides and translocation across membranes. They are induced by cellular stress and protect intracellular proteins by binding and preventing denaturation. In addition, heat shock proteins also play key roles in antigen processing. It has been postulated that heat shock proteins found outside a cell are recognized as a danger signal, indicating to the immune system the presence of damaged or diseased tissue.

Like other heat shock proteins, ORP150 polypeptides function as chaperones that support protein folding and translocation across membranes under physiological conditions. However, expression of ORP150 polypeptides is stress-dependent and preferentially induced by hypoxia, resulting in the accumulation of ORP150 polypeptide in the endoplasmic reticulum (ER). Suppression of ORP150 polypeptide is associated with accelerated apoptosis. ORP150 polypeptides are also suggested to have an important cytoprotective role in hypoxia-induced cellular perturbation. The amino acid sequence of a human ORP150 polypeptide is shown in SEQ ID NO:17 below.

(SEQ ID NO: 17)

```
                10 20 30 40
MADKVRRQRP RRRVCWALVA VLLADLLALS DTLAVMSVDL
                50 60 70 80
GSESMKVAIV KPGVPMEIVL NKESRRKTPV IVILKENERE
                90 100 110 120
FGDSAASMAI KNPKATLRYF QHLLGKQADN PHVALYQARF
              130 140 150 160
PEHELTFDPQ RQTVHFQISS QLQFSPEEVL GMVLNYSRSL
              170 180 190 200
AEDFAEQPIK DAVITVPVFF NQAERRAVLQ AARMAGLKVL
              210 220 230 240
QLINDNTATA LSYGVFRRKD INTTAQNIMF YDMGSGSTVC
              250 260 270 280
TIVTYQMVKT KEAGMQPQLQ IRGVGFDRTL GGLEMELRLR
              290 300 310 320
ERLAGLFNEQ RKGQRAKDVR ENPRAMAKLL REANRLKTVL
              330 340 350 360
SANADHMAQI EGLMDDVDFK AKVTRVEFEE LCADLFERVP
              370 380 390 400
GPVQQALQSA EMSLDEIEQV ILVGGATRVP RVQEVLLKAV
              410 420 430 440
GKEELGKNIN ADEAAAMGAV YQAAALSKAF KVKPFVVRDA
              450 460 470 480
VVYPILVEFT REVEEEPGIH SLKHNKRVLF SRMGPYPQRK
              490 500 510 520
VITFNRYSHD FNFHINYGDL GFLGPEDLRV FGSQNLTTVK
              530 540 550 560
LKGVGDSFKK YPDYESKGIK AHFNLDESGV LSLDRVESVF
              570 580 590 600
ETLVEDSAEE ESTLTKLGNT ISSLFGGGTT PDAKENGTDT
              610 620 630 640
VQEEEESPAE GSKDEPGEQV ELKEEAEAPV EDGSQPPPPE
              650 660 670 680
PKGDATPEGE KATEKENGDK SEAQKPSEKA EAGPEGVAPA
              690 700 710 720
PEGEKKQKPA RKRRMVEEIG VELVVLDLPD LPEDKLAQSV
              730 740 750 760
QKLQDLTLRD LEKQEREKAA NSLEAFIFET QDKLYQPEYQ
              770 780 790 800
EVSTEEQREE ISGKLSAAST WLEDEGVGAT TVMLKEKLAE
              810 820 830 840
LRKLCQGLFF RVEERKKWPE RLSALDNLLN HSSMFLKGAR
              850 860 870 880
LIPEMDQIFT EVEMTTLEKV INETWAWKNA TLAEQAKLPA
              890 900 910 920
TEKPVLLSKD IEAKMMALDR EVQYLLNKAK FTKPRPRPKD
              930  940 950 960
KNGTRAEPPL NASASDQGEK VIPPAGQTED AEPISEPEKV
              970 980 990
ETGSEPGDTE PLELGGPGAE PEQKEQSTGQ KRPLKNDEL
```

Antibodies and Polypeptides that Specifically Bind to an ORP150 Polypeptide Expressed on the Cell Surface of Plasmacytoma, Multiple Myeloma, Colorectal Cancer Cells, Gastric Cancer Cell, or Esophageal Cancer Cells

The invention provides isolated antibodies, and polypeptides derived from the antibodies, that specifically bind to an ORP150 polypeptide expressed on the cell surface of a plasmacytoma cell, multiple myeloma cell, colorectal cancer cell, gastric cancer cell, or esophageal cancer cells (such as a human ORP150 expressed on human cancer cells). A striking feature of such antibodies is their ability to effectively induce plasmacytoma, multiple myeloma, and gastric cancer cell death. As described further in the Examples, antibody 5F4 induced apoptosis and complement dependent cytotoxicity in human plasmacytoma cell lines U266, RPMI 8226, NCI-H929, and L363. Given that these antibodies induce plasmacytoma cell death, they are expected to also induce multiple myeloma cell death. In particular, a plasmacytoma is a solitary myeloma occurring either in the bone or soft tissue. When patients with such localized disease are followed, progression to classic multiple myeloma becomes manifest in most patients with osseous (bone) plasmacytoma, whereas extraosseous (soft tissue) tumors disseminate in only a minor fraction of patients. Multiple myeloma cells are a plasma cell neoplasma that is characterized by involvement of the skeleton at multiple sites. Both plasmacytoma and multiple myeloma are neoplasms of plasma cells, and the clinical treatment for both diseases is basically the same. Thus, the ability of an antibody to induce apoptosis and/or complement dependent cytotoxicity in plasmacytoma cells may indicate that the antibody also induces apoptosis and/or complement dependent cytotoxicity in multiple myeloma cells since they are the same kind of cancer cells (which may differ in the number of lesions involved).

Antibody 5F4, 3B6.1, 6A4.28, and 9A6.2 also bound to colorectal cancer cell lines Colo205, DLD-1, and HT29, gastric cancer cell lines SNU-1 and Kato-III, and esophageal cancer cell line CE146T. Since normal cells lack expression of ORP150 on the cell surface, 5F4 and other antibodies with similar properties have great therapeutic potential treating, preventing, or delaying the development of plasmacytoma, multiple myeloma, colorectal cancer, gastric cancer, and/or esophageal cancer cell. Additionally, 5F4 does not bind to human embryonic vein endothelial cells (HUVEC) or peripheral blood cells, including T-lymphocytes, B-lymphocytes, monocytes, neutrophils, platelets, and red blood cells. Thus, the selectivity for binding to the cell surface of plasmacytoma, multiple myeloma, colorectal cancer cells, gastric cancer cells, and/or esophageal cancer cells minimizes adverse side-effects due to the binding of healthy cells in an individual being treated with an antibody. Such antibodies are also useful for diagnosing plasmacytoma, multiple myeloma, colorectal cancer, gastric cancer, or esophageal cancer.

The antibodies and polypeptides of the invention may optionally have one or more of the following characteristics: (a) exhibit reduced binding to an ORP150 polypeptide (such as binding to an epitope in an extracellular domain of the ORP150 polypeptide) in the presence of antibody 5F4, 3B6.1, 6A4.28, or 9A6.2 (since these antibodies may compete for binding to the ORP polypeptide); (b) induce death of a plasmacytoma, multiple myeloma, or gastric cancer cell (such as through apoptosis) after binding to the ORP150 polypeptide expressed on the cell surface of the cancer cell in the absence of cytotoxin conjugation and immune effector function; (c) induce complement-dependent cytotoxicity in a plasmacytoma, multiple myeloma, colorectal, gastric, or esophageal cancer cell after binding to the cell surface of the cell, (d) induce antibody-dependent cell-mediated cytotoxicity in a plasmacytoma, multiple myeloma, colorectal, gastric, or esophageal cancer cell after binding to the cell surface of the cell, (e) inhibit cell growth or proliferation of a plasmacytoma, multiple myeloma, colorectal, gastric, or esophageal cancer cell after binding to the ORP150 polypeptide expressed on the cell surface of the cancer cell; (f) treat, delay the development of, or prevent plasmacytoma, multiple myeloma, colorectal, gastric, or esophageal cancer expressing the ORP150 polypeptide on the cell surface in an individual; and (g) do not specifically bind one or more of the following cells (e.g., human cells): embryonic vein endothelial cells or peripheral blood cells, such as T-lymphocytes, B-lymphocytes, monocytes, neutrophils, platelets, or red blood cells. In some embodiments, the antibodies and polypeptides bind to human ORP150 shown in SEQ ID NO:17. In some embodiments, the antibodies and polypeptides bind to an epitope within amino acids 723-732, 673-752, 701-800, 673-800 of SEQ ID NO:17.

As used herein, the term “inhibition” includes partial and complete inhibition. For example, binding of the antibody or the polypeptide to an ORP150 polypeptide expressed on the cell surface of a plasmacytoma cell, multiple myeloma cell, colorectal cancer cell, gastric cancer cell, or esophageal cancer cell is inhibited by greater than or about 20%, greater than or about 30%, greater than or about 40%, greater than or about 50%, greater than or about 60%, greater than or about 70%, greater than or about 80%, or greater than or about 90% by an antibody, such as 5F4, described in the Examples. Binding of the antibody to the ORP150 polypeptide (such as an epitope in an extracellular domain of the ORP150 polypeptide) may be inhibited by direct competition or by other mechanisms.

Examples of cancer cells expressing the epitope include, but are not limited to, RPMI8226, U266, NCI-H929, L363, Colo205, DLD-1, HT29, SNU-1, Kato-III, and CE146T cells.

Anti-Idiotypic Antibodies and Polypeptides the Specifically Bind to an Antibody or Polypeptide that Recognizes an ORP150 Polypeptide Expressed on Plasmacytoma, Multiple Myeloma, Colorectal Cancer Cells, Gastric Cancer Cells or Esophageal Cancer Cells

The invention also provides isolated anti-idiotypic antibodies, and polypeptides derived from the anti-idiotypic antibodies, that specifically bind to a primary antibody (or a polypeptide derived from the primary antibody) that specifically binds an ORP150 polypeptide expressed on the cell surface of a plasmacytoma cell, multiple myeloma cell, colorectal cancer cell, gastric cancer cell, or esophageal cancer cell. The anti-idiotypic antibodies and polypeptides of the invention may optionally have one or more of the following characteristics: (a) stimulate an immune response to a plasmacytoma, multiple myeloma, colorectal, gastric, or esophageal cancer cell expressing a ORP150 polypeptide on the cell surface, (b) inhibit cell growth or proliferation of a plasmacytoma, multiple myeloma, colorectal, gastric, or esophageal cancer cell expressing a ORP150 polypeptide on the cell surface; and (c) treat, delay the development of, or prevent plasmacytoma, multiple myeloma, colorectal, gastric, or esophageal cancer expressing a ORP150 polypeptide on the cell surface in an individual.

In particular, one approach toward manipulating the immune response to a tumor-associated antigen is based on idiotypic interactions (U.S. Pat. No. 6,042,827). The unique antigenic determinants in and around the antigen combining site (paratope) of an immunoglobulin molecule are known as idiotopes, and the sum of all idiotopes present on the variable portion of a given antibody is referred to as its idiotype. Idiotypes are serologically defined, since administration of a primary antibody which binds an epitope of the antigen of interest (such as an ORP150 polypeptide expressed on the cell surface of a plasmacytoma cell, multiple myeloma cell, colorectal cancer cell, gastric cancer cell, or esophageal cancer cell) may induce the production of anti-idiotypic antibodies.

When the binding between a primary antibody and an anti-idiotypic antibody is inhibited by the antigen (such as an ORP150 polypeptide expressed on the cell surface of a plasmacytoma cell, multiple myeloma cell, colorectal, gastric, or esophageal cancer cell, or a polypeptide comprising amino acids 673-752, 723-732, 701-800, or 673-800 of SEQ ID NO:17) to which the primary antibody is directed, the idiotype is considered to be binding-site related. In essence, the anti-idiotypic antibody recognizes a paratope-associated idiotope of the primary antibody. Since both the anti-idiotypic antibody and antigen bind to primary antibody, the anti-idiotypic antibody and antigen may share a similar three-dimensional conformation which represents the so-called “internal image” of the epitope. There may be reactions between the primary antibody and other anti-idiotypic antibodies which are not inhibited by antigen, which may involve idiotopes of primary antibody which are spatially distinct from the paratope binding site, yet are still capable of regulating the immune response.

Anti-idiotypic antibodies which act as internal images of a tumor antigen may be used to prime a de novo response to the tumor antigen (such as an ORP150 polypeptide expressed on the cell surface of a plasmacytoma cell, multiple myeloma cell, colorectal, gastric, or esophageal cancer cell, or a polypeptide comprising amino acids 673-800, 701-800, 673-752, or 723-732 of SEQ ID NO:17). By presenting these images of antigenic epitopes in a different molecular environment, responses may be activated which would otherwise be silent. That is, when the anti-idiotype represents the conformational mirror-image of the antigen, it may substitute for nominal antigen and elicit a primary antibody-like response. Additionally, the anti-idiotypic antibody which mimics the antigen may also select or amplify any pre-existing antitumor repertoire by the up regulation of a normally suppressed response.

Anti-idiotypic antibodies which do not bear the internal image of antigen may also induce antitumor responses by influencing the regulatory idiotypic network. Thus, antibodies to framework-associated idiotopes, or regulatory idiotopes, may select or amplify T-cell and/or B-cell clones with specificity for tumor antigens (such as an ORP150 polypeptide expressed on the cell surface of a plasmacytoma cell, multiple myeloma cell, colorectal, gastric, or esophageal cancer cell, or a polypeptide comprising amino acids 673-800, 701-800, 673-752, or 723-732 of SEQ ID NO:17). In some embodiments, an internal image anti-idiotypic antibody and an antibody to one or more framework-associated idiotopes are administered to an individual.

In some embodiments, the combination of an anti-idiotypic antibody and an antigen (such as an ORP150 polypeptide or fragment thereof expressed on the cell surface of a plasmacytoma cell, multiple myeloma cell, colorectal, gastric, or esophageal cancer cell, or a polypeptide comprising amino acids 673-800, 701-800, 673-752, or 723-732 of SEQ ID NO:17) are administered to generate a desired antitumor response.

### Exemplary Embodiments of the Antibodies and Polypeptides of the Invention

The antibodies of the invention can encompass monoclonal antibodies, polyclonal antibodies, antibody fragments (e.g., Fab, Fab′, F(ab′)₂, Fv, Fc, etc.), chimeric antibodies, single chain (ScFv), bispecific antibodies, mutants thereof, fusion proteins comprising an antibody portion, and any other modified configuration of the immunoglobulin molecule that comprises an antigen recognition site of the required specificity. The antibodies may be murine, rat, camel, human, or any other origin (including humanized antibodies).

In some embodiments, the binding affinity of the polypeptide (such as an antibody) to an ORP150 polypeptide is less than or about 500 nM, less than or about 400 nM, less than or about 300 nM, less than or about 200 nM, less than or about 100 nM, less than or about 50 nM, less than or about 10 nM, less than or about 1 nM, less than or about 500 pM, less than or about 100 pM, or less than or about 50 pM. In some embodiments, the binding affinity of an anti-idiotypic antibody (or a polypeptide derived therefrom) to an primary antibody (such as an antibody that specifically binds an ORP150 polypeptide expressed on the cell surface of a plasmacytoma cell, multiple myeloma cell, colorectal cancer cell, gastric cancer cell, or esophageal cancer cell, or an antibody that specifically binds to a polypeptide comprising amino acids 673-800, 701-800, 673-752, or 723-732 of SEQ ID NO:17) is less than or about 500 nM, less than or about 400 nM, less than or about 300 nM, less than or about 200 nM, less than or about 100 nM, less than or about 50 nM, less than or about 10 nM, less than or about 1 nM, less than or about 500 pM, less than or about 100 pM, or less than or about 50 pM. As is well known in the art, binding affinity can be expressed as K_{D}, or dissociation constant, and an increased binding affinity corresponds to a decreased K_{D}. One way of determining binding affinity of antibodies is by measuring binding affinity of monofunctional Fab fragments of the antibody. To obtain monofunctional Fab fragments, an antibody (for example, IgG) can be cleaved with papain or expressed recombinantly. The affinity of an Fab fragment of an antibody can be determined by surface plasmon resonance (BIAcore3000™ surface plasmon resonance (SPR) system, BIAcore, INC, Piscaway N.J.) and ELISA. Kinetic association rates (kₒₙ) and dissociation rates (k_{off}) (generally measured at 25° C.) are obtained; and equilibrium dissociation constant (K_{D}) values are calculated as k_{off}/kₒₙ. In some embodiments, the antibody or polypeptide has an binding affinity for ORP150 that is greater than or about 2, greater than or about 3, greater than or about 5, greater than or about 10, greater than or about 20, greater than or about 50, greater than or about 75, or greater than or about 100-fold more than (strong affinity) than its binding affinity for one or more of the following cells: embryonic vein endothelial cells or peripheral blood cells, such as T-lymphocytes, B-lymphocytes, monocytes, neutrophils, platelets, or red blood cells.

In some embodiments, the antibodies and polypeptides of the invention reduce the number of cancer cells and/or inhibit cell growth or proliferation of tumor or cancer cells (e.g., plasmacytoma cells, multiple myeloma cells, colorectal cancer cells, gastric cancer cell, or esophageal cancer cells) that express an ORP150 polypeptide on the cell surface. Desirably, the reduction in cell number or inhibition of cell growth or proliferation is greater than or about 10%, greater than or about 20%, greater than or about 30%, greater than or about 40%, greater than or about 50%, greater than or about 65%, greater than or about 75%, or greater as compared to the cell not treated with the antibody or polypeptides. In some embodiments, the reduction in cell number or inhibition of cell growth or proliferation due to an antibody or polypeptide of the invention is greater than or about 10%, greater than or about 20%, greater than or about 30%, greater than or about 40%, greater than or about 50%, greater than or about 65%, greater than or about 75%, greater than or about 80%, greater than or about 90%, greater than or about 95%, or greater than or about 100% of the reduction in cell number or inhibition of cell growth or proliferation due to antibody 5F4 under the same conditions. Reduction in cell number or inhibition of cell growth or proliferation can be measured in vitro or in vivo using methods known in the art.

In some embodiments, the antibodies and polypeptides of the invention are capable of inducing cell death alone, for example through apoptosis, after binding the ORP150 polypeptide (such as an epitope in an extracellular domain of the ORP150 polypeptide) expressed on the cell surface of the plasmacytoma, multiple myeloma, or gastric cancer cell. The term “induce cell death” as used herein means that the antibodies or polypeptides of the present invention can directly interact with a molecule expressed on the cell surface, and the binding/interaction alone is sufficient to induce cell death in the cells without the help of other factors such as cytotoxin conjugation or other immune effector functions, i.e., complement-dependent cytotoxicity (CDC), antibody-dependent cellular cytotoxicity (ADCC), or phagocytosis.

As used herein, the term “apoptosis” refers to gene-directed process of intracellular cell destruction. Apoptosis is distinct from necrosis; it includes cytoskeletal disruption, cytoplasmic shrinkage and condensation, expression of phosphatidylserine on the outer surface of the cell membrane and blebbing, resulting in the formation of cell membrane bound vesicles or apoptotic bodies. The process is also referred to as “programmed cell death.” During apoptosis, characteristic phenomena such as curved cell surfaces, condensation of nuclear chromatin, fragmentation of chromosomal DNA, and loss of mitochondrial function are observed. Various known technologies may be used to detect apoptosis, such as staining cells with Annexin V, propidium iodide, DNA fragmentation assay, and/or YO-PRO-1 (Invitrogen).

Methods of detecting cell death (such as apoptosis) in vitro include, but are not limited to, detecting morphology, DNA fragmentation, enzymatic activity, and polypeptide degradation, etc. See Siman et al., U.S. Pat. No. 6,048,703; Martin and Green (1995), Cell, 82: 349-52; Thomberry and Lazebnik (1998), Science, 281:1312-6; Zou et al., U.S. Pat. No. 6,291,643; Scovassi and Poirier (1999), Mol. Cell Biochem., 199: 125-37; Wyllie et al. (1980), Int. Rev. Cytol., 68:251-306; Belhocine et al. (2004), Technol. Cancer Res. Treat., 3(1):23-32, which are incorporated herein by reference.

In some embodiments, the antibodies and polypeptides of the invention specifically bind to a polypeptide comprising amino acids 673-800, 701-800, 673-752, or 723-732 of SEQ ID NO:17.

In some embodiments, the antibodies and polypeptides of the invention compete with antibody 5F4, 3B6.1, 6A4.28, or 9A6.2 for binding to an ORP150 polypeptide (such as an epitope in an extracellular domain of the ORP150 polypeptide) expressed on the cell surface of the cancer cell. In some embodiments, the antibodies or polypeptides of the invention bind to an epitope on an ORP150 polypeptide to which antibody 5F4, 3B6.1, 6A4.28, or 9A6.2 binds.

Competition assays can be used to determine whether two antibodies bind the same epitope by recognizing identical or sterically overlapping epitopes or one antibody competitively inhibits binding of another antibody to the antigen. These assays are known in the art. Typically, antigen or antigen expressing cells is immobilized on a multi-well plate and the ability of unlabeled antibodies to block the binding of labeled antibodies is measured. Common labels for such competition assays are radioactive labels or enzyme labels. For example, immobilized ORP150 polypeptide is incubated with a first labeled antibody that binds to the polypeptide and an increasing concentrations of a second unlabeled antibody. As a control, immobilized ORP150 polypeptide is incubated with the first labeled antibody without the second unlabeled antibody. After incubation under conditions that allow binding the first antibody to the immobilized polypeptide, excess unbound antibody is removed and the amount of label bound to the immobilized polypeptide is measured. If the amount of label bound to the immobilized polypeptide is substantially reduced (for example, reduced at least about 50%, at least about 60%, at least about 70%, at least about 80%, or at least about 90%) in the test sample relative to the control sample when the concentration of the second unlabeled antibody to the first labeled antibody in the test is 100:1 or higher (such as 500:1 or higher, or 1000:1 or higher), the second antibody is considered as competing with the first antibody for binding to the polypeptide. Other methods may be used to for mapping to which an antibody binds are provided in Morris (1996) “Epitope Mapping Protocols,” in Methods in Molecular Biology v. 66 (Humana Press, Totowa, N.J.).

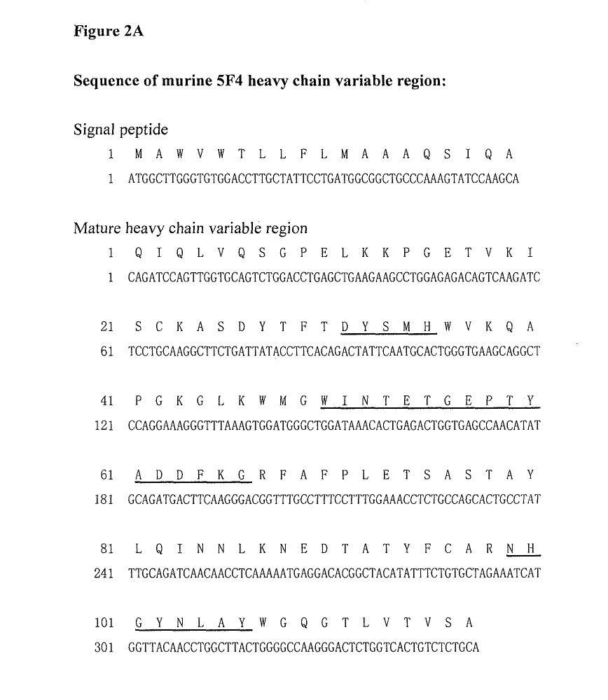

In some embodiments, the antibody of the invention is antibody 5F4 or an antibody derived from 5F4. The invention provides an antibody or a polypeptide comprising a fragment or a region of the antibody 5F4 (such as an antibody or a polypeptide comprising the sequence in FIG. 2A or 2B or a fragment thereof). In one embodiment, the fragment is a light chain of the antibody 5F4. In another embodiment, the fragment is a heavy chain of the antibody 5F4. In yet another embodiment, the fragment contains one or more variable regions from a light chain and/or a heavy chain of the antibody 5F4 (such as an antibody or a polypeptide comprising the sequence in FIG. 2A or 2B or a fragment thereof). In yet another embodiment, the fragment contains one, two, or three CDRs from a light chain and/or a heavy chain of the antibody 5F4 (such as a CDR from the sequence in FIG. 2A or 2B). In some embodiments, the antibody is a humanized version of antibody 5F4. In some embodiments, the one or more CDRs derived from antibody 5F4 are greater than or about 85%, greater than or about 86%, greater than or about 87%, greater than or about 88%, greater than or about 89%, greater than or about 90%, greater than or about 91%, greater than or about 92%, greater than or about 93%, greater than or about 94%, greater than or about 95%, greater than or about 96%, greater than or about 97%, greater than or about 98%, or greater than or about 99% identical to one or more, two or more, three or more, four or more, five or more, or six CDRs of antibody 5F4 (such as a CDR from the sequence in FIG. 2A or 2B). In some embodiments, the CDR is a Kabat CDR. In other embodiments, the CDR is a Chothia CDR. In other embodiments, the CDR is a combination of a Kabat and a Chothia CDR (also termed “combined CDR” or “extended CDR”). In other words, for any given embodiment containing more than one CDR, the CDRs may be any of Kabat, Chothia, and/or combined. In some embodiments, the antibody or a polypeptide consists of or comprises the amino acid sequence of SEQ ID NO: 1 and/or 3.

In some embodiments, the antibody of the invention is antibody h5F4Ac.2/v17 in IgG1, IgG2, IgG3, or IgG4 isotype or an antibody derived from the variable regions of h5F4Ac.2/v17. In some embodiments, the invention provides an antibody or a polypeptide comprising the sequence in SEQ ID NO:19 or 20 or a fragment thereof. In one embodiment, the fragment is a light chain of the antibody h5F4Ac.2/v17. In another embodiment, the fragment is a heavy chain of the antibody h5F4Ac.2/v17. In yet another embodiment, the fragment contains one or more variable regions from a light chain having the amino acid sequence of SEQ ID NO:20 and/or a heavy chain having the amino acid sequence of SEQ ID NO:19. In yet another embodiment, the fragment contains one, two, or three CDRs from a light chain and/or a heavy chain of the antibody h5F4Ac.2/v17 (such as a CDR from the sequence in SEQ ID NO:20 or 19). In some embodiments, the antibody is a humanized antibody. In some embodiments, the one or more CDRs derived from h5F4Ac.2/v17 are greater than or about 85%, greater than or about 86%, greater than or about 87%, greater than or about 88%, greater than or about 89%, greater than or about 90%, greater than or about 91%, greater than or about 92%, greater than or about 93%, greater than or about 94%, greater than or about 95%, greater than or about 96%, greater than or about 97%, greater than or about 98%, or greater than or about 99% identical to one or more, two or more, three or more, four or more, five or more, or six CDRs of antibody h5F4Ac.2/v17 (such as a CDR from the sequence in SEQ ID NO:19 or 20). In some embodiments, the CDR is a Kabat CDR. In other embodiments, the CDR is a Chothia CDR. In other embodiments, the CDR is a combination of a Kabat and a Chothia CDR (also termed “combined CDR” or “extended CDR”). In other words, for any given embodiment containing more than one CDR, the CDRs may be any of Kabat, Chothia, and/or combined. In some embodiments, the antibody or a polypeptide consists of or comprises the amino acid sequence of SEQ ID NO:19 and/or 20.

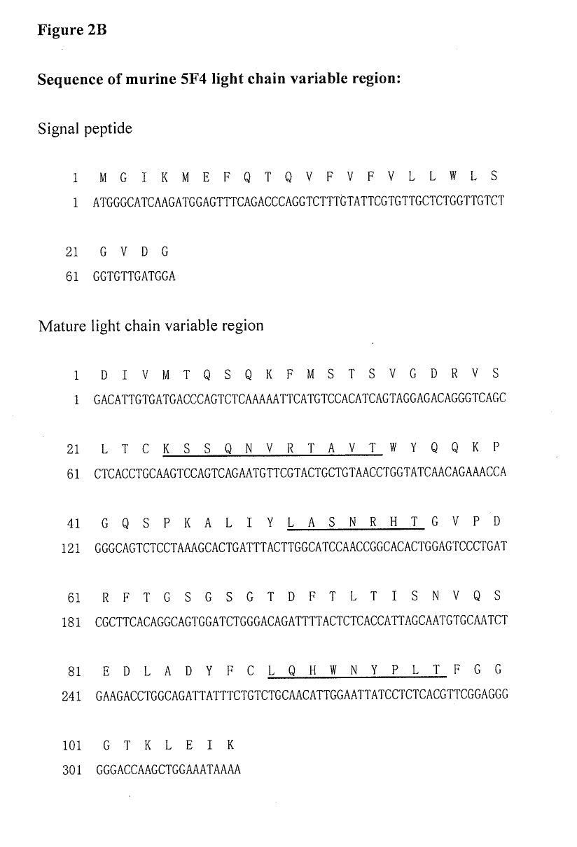

In some embodiments, the antibody of the invention is antibody 3B6.1 or an antibody derived from 3B6.1. The invention provides an antibody or a polypeptide comprising a fragment or a region of the antibody 3B6.1 (such as an antibody or a polypeptide comprising the sequence in FIG. 3A or 3B or a fragment thereof). In one embodiment, the fragment is a light chain of the antibody 3B6.1. In another embodiment, the fragment is a heavy chain of the antibody 3B6.1. In yet another embodiment, the fragment contains one or more variable regions from a light chain and/or a heavy chain of the antibody 3B6.1 (such as an antibody or a polypeptide comprising the sequence in FIG. 3A or 3B or a fragment thereof). In yet another embodiment, the fragment contains one, two, or three CDRs from a light chain and/or a heavy chain of the antibody 3B6.1 (such as a CDR from the sequence in FIG. 3A or 3B). In some embodiments, the antibody is a humanized version of antibody 5F4. In some embodiments, the one or more CDRs derived from antibody 3B6.1 are greater than or about 85%, greater than or about 86%, greater than or about 87%, greater than or about 88%, greater than or about 89%, greater than or about 90%, greater than or about 91%, greater than or about 92%, greater than or about 93%, greater than or about 94%, greater than or about 95%, greater than or about 96%, greater than or about 97%, greater than or about 98%, or greater than or about 99% identical to one or more, two or more, three or more, four or more, five or more, or six CDRs of antibody 3B6.1 (such as a CDR from the sequence in FIG. 3A or 3B). In some embodiments, the CDR is a Kabat CDR. In other embodiments, the CDR is a Chothia CDR. In other embodiments, the CDR is a combination of a Kabat and a Chothia CDR (also termed “combined CDR” or “extended CDR”). In other words, for any given embodiment containing more than one CDR, the CDRs may be any of Kabat, Chothia, and/or combined. In some embodiments, the antibody or a polypeptide consists of or comprises the amino acid sequence of SEQ ID NO:5 and/or 7.

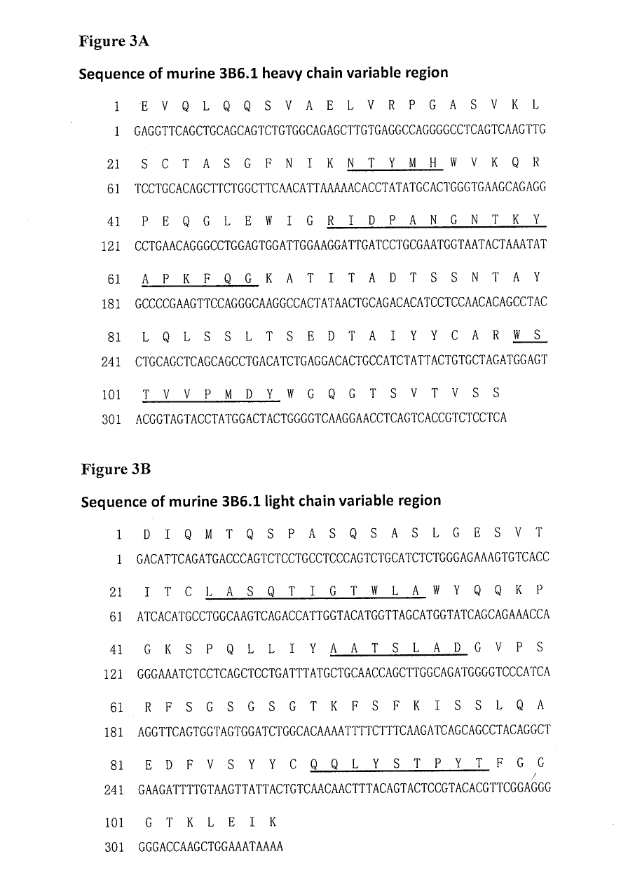

In some embodiments, the antibody of the invention is antibody 6A4.28 or an antibody derived from 6A4.28. The invention provides an antibody or a polypeptide comprising a fragment or a region of the antibody 6A4.28 (such as an antibody or a polypeptide comprising the sequence in FIG. 4A or 4B or a fragment thereof). In one embodiment, the fragment is a light chain of the antibody 6A4.28. In another embodiment, the fragment is a heavy chain of the antibody 6A4.28. In yet another embodiment, the fragment contains one or more variable regions from a light chain and/or a heavy chain of the antibody 6A4.28 (such as an antibody or a polypeptide comprising the sequence in FIG. 4A or 4B or a fragment thereof). In yet another embodiment, the fragment contains one, two, or three CDRs from a light chain and/or a heavy chain of the antibody 6A4.28 (such as a CDR from the sequence in FIG. 4A or 4B). In some embodiments, the antibody is a humanized version of antibody 6A4.28. In some embodiments, the one or more CDRs derived from antibody 6A4.28 are greater than or about 85%, greater than or about 86%, greater than or about 87%, greater than or about 88%, greater than or about 89%, greater than or about 90%, greater than or about 91%, greater than or about 92%, greater than or about 93%, greater than or about 94%, greater than or about 95%, greater than or about 96%, greater than or about 97%, greater than or about 98%, or greater than or about 99% identical to one or more, two or more, three or more, four or more, five or more, or six CDRs of antibody 6A4.28 (such as a CDR from the sequence in FIG. 4A or 4B). In some embodiments, the CDR is a Kabat CDR. In other embodiments, the CDR is a Chothia CDR. In other embodiments, the CDR is a combination of a Kabat and a Chothia CDR (also termed “combined CDR” or “extended CDR”). In other words, for any given embodiment containing more than one CDR, the CDRs may be any of Kabat, Chothia, and/or combined. In some embodiments, the antibody or a polypeptide consists of or comprises the amino acid sequence of SEQ ID NO:9 and/or 11.

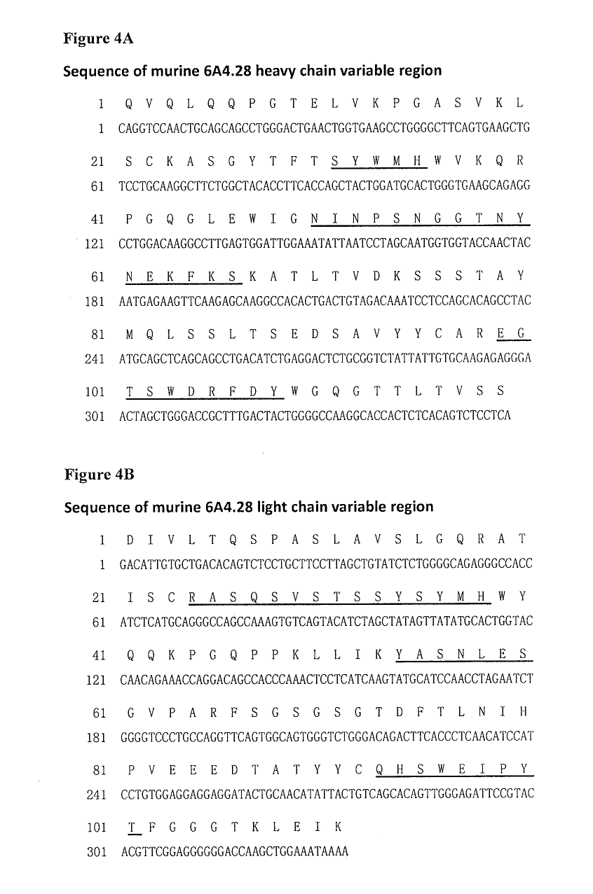

In some embodiments, the antibody of the invention is antibody 9A6.2 or an antibody derived from 9A6.2. The invention provides an antibody or a polypeptide comprising a fragment or a region of the antibody 9A6.2 (such as an antibody or a polypeptide comprising the sequence in FIG. 5A or 5B or a fragment thereof). In one embodiment, the fragment is a light chain of the antibody 9A6.2. In another embodiment, the fragment is a heavy chain of the antibody 9A6.2. In yet another embodiment, the fragment contains one or more variable regions from a light chain and/or a heavy chain of the antibody 9A6.2 (such as an antibody or a polypeptide comprising the sequence in FIG. 5A or 5B or a fragment thereof). In yet another embodiment, the fragment contains one, two, or three CDRs from a light chain and/or a heavy chain of the antibody 9A6.2 (such as a CDR from the sequence in FIG. 5A or 5B). In some embodiments, the antibody is a humanized version of antibody 5F4. In some embodiments, the one or more CDRs derived from antibody 9A6.2 are greater than or about 85%, greater than or about 86%, greater than or about 87%, greater than or about 88%, greater than or about 89%, greater than or about 90%, greater than or about 91%, greater than or about 92%, greater than or about 93%, greater than or about 94%, greater than or about 95%, greater than or about 96%, greater than or about 97%, greater than or about 98%, or greater than or about 99% identical to one or more, two or more, three or more, four or more, five or more, or six CDRs of antibody 9A6.2 (such as a CDR from the sequence in FIG. 5A or 5B). In some embodiments, the CDR is a Kabat CDR. In other embodiments, the CDR is a Chothia CDR. In other embodiments, the CDR is a combination of a Kabat and a Chothia CDR (also termed “combined CDR” or “extended CDR”). In other words, for any given embodiment containing more than one CDR, the CDRs may be any of Kabat, Chothia, and/or combined. In some embodiments, the antibody or a polypeptide consists of or comprises the amino acid sequence of SEQ ID NO:13 and/or 15.

Methods of making antibodies and polypeptides derived from the antibodies are known in the art and are disclosed herein. The antibodies of the present invention can be prepared using well-established methods. For example, the monoclonal antibodies can be prepared using hybridoma technology, such as those described by Kohler and Milstein (1975), Nature, 256:495. In a hybridoma method, a mouse, a hamster, or other appropriate host animal, is typically immunized with an immunizing agent (e.g., a cancer cell expressing an ORP150 polypeptide, an ORP150 polypeptide, or an extracellular domain or fragment thereof of an ORP150 polypeptide expressed by the cancer cell, which may be purified using antibodies described herein; or a polypeptide comprising amino acids 673-800, 701-800, 673-752, or 723-732 of SEQ ID NO:17) to elicit lymphocytes that produce or are capable of producing antibodies that specifically bind to the immunizing agent. Alternatively, the lymphocytes may be immunized in vitro. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-1031). Immortalized cell lines are usually transformed mammalian cells, particularly myeloma cells of rodent, rabbit, bovine, or human origin. Usually, rat or mouse myeloma cell lines are employed. The hybridoma cells may be cultured in a suitable culture medium that desirably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically includes hypoxanthine, aminopterin, and thymidine (“HAT medium”), which substances prevent the growth of HGPRT-deficient cells.

Desired immortalized cell lines are those that fuse efficiently, support stable high level expression of antibody by the selected antibody-producing cells, and are sensitive to a medium such as HAT medium. More desirable immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol. (1984), 133:3001; Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies. The antibody may be screened for having specific binding to an ORP150 polypeptide (such as binding to an epitope in an extracellular domain of the ORP150 polypeptide) obtained from or expressed on the cell surface of plasmacytoma, multiple myeloma, colorectal, gastric, or esophageal cancer or tumor cells. Cancer cells or an ORP150 polypeptide (or a fragment thereof containing an extracellular domain of an ORP150 polypeptide) may be used for screening. For example, RPMI8226, U266, NCI-H929, L363, Colo205, DLD-1, HT29, SNU-1, Kato-III, or CE146T cells may be used for screening. A polypeptide comprising amino acids 673-800, 701-800, 673-752, or 723-732 of SEQ ID NO:17 may also be used for screening.

In some embodiments, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard (1980), Anal. Biochem., 107:220.

After the desired hybridoma cells are identified, the clones may be subcloned by limiting dilution procedures and grown by standard methods (Goding, supra). Suitable culture media for this purpose include, for example, Dulbecco's Modified Eagle's Medium or RPMI-1640 medium. Alternatively, the hybridoma cells may be grown in vivo as ascites in a mammal.

The monoclonal antibodies can be generated by culturing the hybridoma cells, and the antibodies secreted by the hybridoma cells may further be isolated or purified. Antibodies may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The antibodies or polypeptides of the invention may be generated by screening a library of antibodies or polypeptides to select antibodies or polypeptides that bind to an ORP150 polypeptide expressed on the cell surface of a plasmacytoma cell, a multiple myeloma cell, a colorectal cancer cell, a gastric cancer cell, or an esophageal cancer cell. For example, binding of the antibodies or polypeptides to a polypeptide comprising amino acids 673-800, 701-800, 673-752, or 723-732 of SEQ ID NO:17, and/or a plasmacytoma, multiple myeloma, colorectal, gastric, and/or esophageal cancer cell expressing an ORP150 polypeptide on the cell surface may be tested. Antibody phage display libraries known in the art may be used. In some embodiments, the antibodies in the library (e.g., displayed on phage) are single-chain Fv (scFv) fragments or Fab fragment. In some embodiments, the antibodies in the library (e.g., displayed on phage) are single-domain antibodies. For example, a single-domain antibody may comprise all or a portion of the heavy chain variable domain or all or a portion of the light chain variable domain of an antibody. In some embodiments, the antibodies in the library are human antibodies.

The antibodies identified may further be tested for their capabilities to induce cell death (e.g., apoptosis), and/or inhibit cell growth or proliferation of a plasmacytoma, multiple myeloma, colorectal, gastric, and/or esophageal cancer cell using methods known in the art and described herein.

The antibodies of the invention can also be made by recombinant DNA methods, such as those described in U.S. Pat. Nos. 4,816,567 and 6,331,415, which are hereby incorporated by reference. For example, DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein to synthesize monoclonal antibodies in the recombinant host cells. The DNA also can be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

In some embodiment, the antibodies of the present invention are expressed from two expression vectors. The first expression vector encodes a heavy chain of the antibody (e.g., a humanized antibody) and comprises a first part encoding a variable region of the heavy chain of the antibody, and a second part encoding a constant region of the heavy chain of the antibody. The second expression vector encodes a light chain of the antibody and comprises a first part encoding a variable region of the light chain of the antibody, and a second part encoding a constant region of the light chain of the antibody.

Alternatively, the antibodies (e.g., a humanized antibody) of the present invention are expressed from a single expression vector. The single expression vector encodes both the heavy chain and light chain of the antibodies of the present invention. In some embodiments, the expression vector comprises a polynucleotide sequence encoding a variable region of the heavy chain and a variable region of the light chain of antibody 5F4, 3B6.1, 6A4.28, or 9A6.2.

Normally the expression vector has transcriptional and translational regulatory sequences which are derived from a species compatible with a host cell. In addition, the vector ordinarily carries a specific gene(s) which is (are) capable of providing phenotypic selection in transformed cells.

A wide variety of recombinant host-vector expression systems for eukaryotic cells are known and can be used in the invention. For example, Saccharomyces cerevisiae, or common baker's yeast, is the most commonly used among eukaryotic microorganisms, although a number of other strains, such as Pichia pastoris, are available. Cell lines derived from multicellular organisms such as Sp2/0 or Chinese Hamster Ovary (CHO), which are available from the ATCC, may also be used as hosts. Typical vector plasmids suitable for eukaryotic cell transformations are, for example, pSV2neo and pSV2gpt (ATCC), pSVL and pSVK3 (Pharmacia), and pBPV-1/pML2d (International Biotechnology, Inc.).

The eukaryotic host cells useful in the present invention are, for example, hybridoma, myeloma, plasmacytoma, or lymphoma cells. However, other eukaryotic host cells may be suitably utilized provided the mammalian host cells are capable of recognizing transcriptional and translational DNA sequences for expression of the proteins; processing the leader peptide by cleavage of the leader sequence and secretion of the proteins; and providing post-translational modifications of the proteins, e.g., glycosylation.

Accordingly, the present invention provides eukaryotic host cells which are transformed by recombinant expression vectors comprising DNA constructs disclosed herein and which are capable of expressing the antibodies or polypeptides of the present invention. In some embodiments, the transformed host cells of the invention comprise at least one DNA construct comprising the light and heavy chain DNA sequences described herein, and transcriptional and translational regulatory sequences which are positioned in relation to the light and heavy chain-encoding DNA sequences to direct expression of antibodies or polypeptides.

The host cells used in the invention may be transformed in a variety of ways by standard transfection procedures well known in the art. Among the standard transfection procedures which may be used are electroporation techniques, protoplast fusion and calcium-phosphate precipitation techniques. Such techniques are generally described by F. Toneguzzo et al. (1986), Mol. Cell. Biol., 6:703-706; G. Chu et al., Nucleic Acid Res. (1987), 15:1311-1325; D. Rice et al., Proc. Natl. Acad. Sci. USA (1979), 79:7862-7865; and V. Oi et al., Proc. Natl. Acad. Sci. USA (1983), 80:825-829.

In the case of two expression vectors, the two expression vectors can be transferred into a host cell one by one separately or together (co-transfer or co-transfect).

The present invention also provides a method for producing the antibodies or polypeptides that comprises culturing a host cell comprising an expression vector(s) encoding the antibodies or the polypeptides, and recovering the antibodies or polypeptides from the culture by ways well know to one skilled in the art.

Furthermore, the desired antibodies can be produced in a transgenic animal. A suitable transgenic animal can be obtained according to standard methods which include micro-injecting into eggs the appropriate expression vectors, transferring the eggs into pseudo-pregnant females, and selecting a descendant expressing the desired antibody.

The present invention also provides chimeric antibodies that specifically recognize an ORP150 polypeptide (such as an epitope in an extracellular domain of the ORP150 polypeptide) expressed by a cancer cell. For example, the variable and constant regions of the chimeric antibody are from separate species. In some embodiments, the variable regions of both heavy chain and light chain are from the murine antibodies described herein. In some embodiments, the variable regions comprise amino acid sequences of antibody 5F4, 3B6.1, 6A4.28, or 9A6.2. In some embodiments, the constant regions of both the heavy chain and light chain are from human antibodies.

The chimeric antibody of the present invention can be prepared by techniques well-established in the art. See for example, U.S. Pat. No. 6,808,901; U.S. Pat. No. 6,652,852; U.S. Pat. No. 6,329,508; U.S. Pat. No. 6,120,767; and U.S. Pat. No. 5,677,427, each of which is hereby incorporated by reference. In general, the chimeric antibody can be prepared by obtaining cDNAs encoding the heavy and light chain variable regions of the antibodies, inserting the cDNAs into an expression vector, which upon being introduced into eukaryotic host cells, expresses the chimeric antibody of the present invention. Desirably, the expression vector carries a functionally complete constant heavy or light chain sequence so that any variable heavy or light chain sequence can be easily inserted into the expression vector.

The present invention provides a humanized antibody that specifically recognizes an ORP150 polypeptide (such as an epitope in an extracellular domain of the ORP150 polypeptide) expressed by a plasmacytoma, multiple myeloma, colorectal, gastric, or esophageal cancer cell. The humanized antibody is typically a human antibody in which residues from CDRs are replaced with residues from CDRs of a non-human species such as mouse, rat, or rabbit having the desired specificity, affinity and capacity. In some instances, Fv framework residues of the human antibody are replaced by corresponding non-human residues.

There are four general steps to humanize a monoclonal antibody. These are: (1) determining the nucleotide and predicted amino acid sequence of the starting antibody light and heavy variable domains, (2) designing the humanized antibody, i.e., deciding which antibody framework region to use during the humanizing process, (3) the actual humanizing methodologies/techniques, and (4) the transfection and expression of the humanized antibody. See, for example, U.S. Pat. Nos. 4,816,567; 5,807,715; 5,866,692; 6,331,415; 5,530,101; 5,693,761; 5,693,762; 5,585,089; 6,180,370; and 6,548,640. For example, the constant region may be engineered to more resemble human constant regions to avoid immune response if the antibody is used in clinical trials and treatments in humans. See, for example, U.S. Pat. Nos. 5,997,867 and 5,866,692.

It is important that antibodies be humanized with retention of high affinity for the antigen and other favorable biological properties. To achieve this goal, humanized antibodies can be prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding. The humanized antibodies may also contain modifications in the hinge region to improve one or more characteristics of the antibody.

In another alternative, antibodies may be screened and made recombinantly by phage display technology. See, for example, U.S. Pat. Nos. 5,565,332; 5,580,717; 5,733,743 and 6,265,150; and Winter et al., Annu. Rev. Immunol. 12:433-455 (1994). Alternatively, the phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for review see, e.g., Johnson, Kevin S. and Chiswell, David J., Current Opinion in Structural Biology 3, 564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed, and antibodies to a diverse array of antigens (including self-antigens) can be isolated essentially following the techniques described by Mark et al., J. Mol. Biol. 222:581-597 (1991), or Griffith et al., EMBO J. 12:725-734 (1993). In a natural immune response, antibody genes accumulate mutations at a high rate (somatic hypermutation). Some of the changes introduced will confer higher affinity, and B-cells displaying high-affinity surface immunoglobulin are preferentially replicated and differentiated during subsequent antigen challenge. This natural process can be mimicked by employing the technique known as “chain shuffling.” Marks et al., Bio/Technol. 10:779-783 (1992)). In this method, the affinity of “primary” human antibodies obtained by phage display can be improved by sequentially replacing the heavy and light chain V region genes with repertoires of naturally occurring variants (repertoires) of V domain genes obtained from unimmunized donors. This technique allows the production of antibodies and antibody fragments with affinities in the pM-nM range. A strategy for making very large phage antibody repertoires (also known as “the mother-of-all libraries”) has been described by Waterhouse et al., Nucl. Acids Res. 21:2265-2266 (1993). Gene shuffling can also be used to derive human antibodies from rodent antibodies, where the human antibody has similar affinities and specificities to the starting rodent antibody. According to this method, which is also referred to as “epitope imprinting,” the heavy or light chain V domain gene of rodent antibodies obtained by phage display technique is replaced with a repertoire of human V domain genes, creating rodent-human chimeras. Selection on antigen results in isolation of human variable regions capable of restoring a functional antigen-binding site, i.e., the epitope governs (imprints) the choice of partner. When the process is repeated in order to replace the remaining rodent V domain, a human antibody is obtained (see PCT Publication No. WO 93/06213, published Apr. 1, 1993). Unlike traditional humanization of rodent antibodies by CDR grafting, this technique provides completely human antibodies, which have no framework or CDR residues of rodent origin. It is apparent that although the above discussion pertains to humanized antibodies, the general principles discussed are applicable to customizing antibodies for use, for example, in dogs, cats, primates, equines, and bovines.

In certain embodiments, the antibody is a fully human antibody. Non-human antibodies that specifically bind an antigen can be used to produce a fully human antibody that binds to that antigen. For example, the skilled artisan can employ a chain swapping technique, in which the heavy chain of a non-human antibody is co-expressed with an expression library expressing different human light chains. The resulting hybrid antibodies, containing one human light chain and one non-human heavy chain, are then screened for antigen binding. The light chains that participate in antigen binding are then co-expressed with a library of human antibody heavy chains. The resulting human antibodies are screened once more for antigen binding. Techniques such as this one are further described in U.S. Pat. No. 5,565,332. In addition, an antigen can be used to inoculate an animal that is transgenic for human immunoglobulin genes. See, e.g., U.S. Pat. No. 5,661,016.

The invention also provides bispecific antibodies. A bispecific antibody has binding specificities for at least two different antigens (including different epitopes). In some embodiments, one of the binding specificities of the bispecific antibody is for an epitope on an ORP150 polypeptide expressed on the cell surface of a plasmacytoma cell, multiple myeloma cell, colorectal cancer cell, gastric cancer cell, or esophageal cancer cell. In some embodiments, the bispecific antibody comprises a first binding domain that specifically binds to an ORP150 polypeptide expressed on the cell surface of a plasmacytoma cell, multiple myeloma cell, colorectal cancer cell, gastric cancer cell, or esophageal cancer cell, and a second binding domain that specifically binds to a different antigen. In some embodiments, bispecific antibodies are human or humanized antibodies. In some embodiments, the bispecific antibody comprises a heavy chain variable region comprising one, two, or three CDRs derived from any of the ORP150 antibodies described herein (e.g., 5F4, 3B6.1, 6A4.28, 9A6.2, or h5F4Ac.2/v17) and/or a light chain variable region comprising one, two, or three CDRs derived from any of the ORP150 antibodies described herein (e.g., 5F4, 3B6.1, 6A4.28, 9A6.2, or h5F4Ac.2/v17).

A bispecific antibody (a monoclonal antibody that has binding specificities for at least two different antigens) can be prepared using the antibodies disclosed herein. Methods for making bispecific antibodies are known in the art (see, e.g., Suresh et al., 1986, Methods in Enzymology 121:210). Traditionally, the recombinant production of bispecific antibodies was based on the coexpression of two immunoglobulin heavy chain-light chain pairs, with the two heavy chains having different specificities (Millstein and Cuello, 1983, Nature 305, 537-539).

According to one approach to making bispecific antibodies, antibody variable domains with the desired binding specificities (antibody-antigen combining sites) are fused to immunoglobulin constant domain sequences. In some embodiments, the fusion is with an immunoglobulin heavy chain constant domain, comprising at least part of the hinge, CH2, and CH3 regions. It is preferred to have the first heavy chain constant region (CH1), containing the site necessary for light chain binding, present in at least one of the fusions. DNAs encoding the immunoglobulin heavy chain fusions and, if desired, the immunoglobulin light chain, are inserted into separate expression vectors, and are cotransfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In one approach, the bispecific antibodies are composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations. This approach is described in PCT Publication No. WO 94/04690, published Mar. 3, 1994.

Heteroconjugate antibodies, comprising two covalently joined antibodies, are also within the scope of the invention. Such antibodies have been used to target immune system cells to unwanted cells (U.S. Pat. No. 4,676,980), and for treatment of HIV infection (PCT Publication Nos. WO 91/00360 and WO 92/200373; and EP 03089). Heteroconjugate antibodies may be made using any convenient cross-linking methods. Suitable cross-linking agents and techniques are well known in the art, and are described in U.S. Pat. No. 4,676,980.

Single chain Fv fragments may also be produced, such as described in Iliades et al., 1997, FEBS Letters, 409:437-441. Coupling of such single chain fragments using various linkers is described in Kortt et al., 1997, Protein Engineering, 10:423-433. A variety of techniques for the recombinant production and manipulation of antibodies are well known in the art.

It is contemplated that the present invention encompasses not only the monoclonal antibodies described above, but also any fragments thereof containing the active binding region of the antibodies, such as Fab, F(ab′)₂, scFv, Fv fragments, and the like. Such fragments can be produced from the monoclonal antibodies described herein using techniques well established in the art (Rousseaux et al. (1986), in Methods Enzymol., 121:663-69 Academic Press).

Methods of preparing antibody fragment are well known in the art. For example, an antibody fragment can be produced by enzymatic cleavage of antibodies with pepsin to provide a 100 Kd fragment denoted F(ab′)₂. This fragment can be further cleaved using a thiol reducing agent, and optionally a blocking group for the sulfhydryl groups resulting from cleavage of disulfide linkages, to produce 50 Kd Fab′ monovalent fragments. Alternatively, an enzymatic cleavage using papain produces two monovalent Fab fragments and an Fc fragment directly. These methods are described, for example, by U.S. Pat. Nos. 4,036,945 and 4,331,647 and references contained therein, which patents are incorporated herein by reference. Also, see Nisonoff et al. (1960), Arch Biochem. Biophys. 89: 230; Porter (1959), Biochem. J. 73: 119; Smyth (1967), Methods in Enzymology 11: 421-426.

Alternatively, the Fab can be produced by inserting DNA encoding Fab of the antibody into an expression vector for prokaryote or an expression vector for eukaryote, and introducing the vector into a prokaryote or eukaryote to express the Fab.

The invention encompasses modifications to antibodies or polypeptide described herein, including functionally equivalent antibodies which do not significantly affect their properties and variants which have enhanced or decreased activity and/or affinity. For example, amino acid sequence of antibody 5F4, 3B6.1, 6A4.28, or 9A6.2 or humanized antibody, may be mutated to obtain an antibody with the desired binding affinity to an ORP150 polypeptide expressed by the cancer cell. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope tag. Other insertional variants of the antibody molecule include the fusion to the N- or C-terminus of the antibody of an enzyme or a polypeptide which increases the serum half-life of the antibody.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in the table below under the heading of “conservative substitutions.” If such substitutions result in a change in biological activity, then more substantial changes, denominated “exemplary substitutions” in the table below, or as further described below in reference to amino acid classes, may be introduced and the products screened.

Amino Acid Substitutions

| Original | Conservative | Exemplary |
|---|---|---|
| Residue | Substitutions | Substitutions |
| Ala (A) | Val | Val; Leu; Ile |
| Arg (R) | Lys | Lys; Gln; Asn |
| Asn (N) | Gln | Gln; His; Asp, Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; Phe; |
| | | Norleucine |
| Leu (L) | Ile | Norleucine; Ile; Val; Met; |
| | | Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; Phe; Ala; |
| | | Norleucine |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) Non-polar: Norleucine, Met, Ala, Val, Leu, Ile;

(2) Polar without charge: Cys, Ser, Thr, Asn, Gln;

(3) Acidic (negatively charged): Asp, Glu;

(4) Basic (positively charged): Lys, Arg;

(5) Residues that influence chain orientation: Gly, Pro; and

(6) Aromatic: Trp, Tyr, Phe, His.

Non-conservative substitutions are made by exchanging a member of one of these classes for another class.

Any cysteine residue not involved in maintaining the proper conformation of the antibody also may be substituted, generally with serine, to improve the oxidative stability of the molecule and prevent aberrant cross-linking. Conversely, cysteine bond(s) may be added to the antibody to improve its stability, particularly where the antibody is an antibody fragment such as an Fv fragment.

Amino acid modifications can range from changing or modifying one or more amino acids to complete redesign of a region, such as the variable region. Changes in the variable region can alter binding affinity and/or specificity. In some embodiments, no more than one to five conservative amino acid substitutions are made within a CDR domain. In other embodiments, no more than one to three conservative amino acid substitutions are made within a CDR domain. In still other embodiments, the CDR domain is CDRH3 and/or CDRL3.

In some embodiments, one or more amino acid residues in the heavy chain constant region and/or the light chain constant region of the antibody are modified. For example, amino acid residues of antibodies described in the Examples may be modified. In some embodiments, the Fc region of antibodies is modified to enhance or reduce ADCC and/or CDC activities of the antibodies. See Shields et al., J. Biol. Chem. 276:6591-6604 (2001); Presta et al., Biochem. Soc. Trans. 30:487-490 (2002).

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Hefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Mature Biotech. 17:176-180).

Glycosylation of antibodies is typically either N-linked or O-linked. N-linked refers to the attachment of the carbohydrate moiety to the side chain of an asparagine residue. The tripeptide sequences asparagine-X-serine, asparagine-X-threonine, and asparagine-X-cysteine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

The glycosylation pattern of antibodies may also be altered without altering the underlying nucleotide sequence. Glycosylation largely depends on the host cell used to express the antibody. Since the cell type used for expression of recombinant glycoproteins, e.g., antibodies, as potential therapeutics is rarely the native cell, variations in the glycosylation pattern of the antibodies can be expected (see, e.g., Hse et al., 1997, J. Biol. Chem. 272:9062-9070).

In addition to the choice of host cells, factors that affect glycosylation during recombinant production of antibodies include growth mode, media formulation, culture density, oxygenation, pH, purification schemes, and the like. Various methods have been proposed to alter the glycosylation pattern achieved in a particular host organism including introducing or overexpressing certain enzymes involved in oligosaccharide production (U.S. Pat. Nos. 5,047,335; 5,510,261; and 5,278,299). Glycosylation, or certain types of glycosylation, can be enzymatically removed from the glycoprotein, for example using endoglycosidase H (Endo H), N-glycosidase F, endoglycosidase F1, endoglycosidase F2, or endoglycosidase F3. In addition, the recombinant host cell can be genetically engineered to be defective in processing certain types of polysaccharides. These and similar techniques are well known in the art.

In some embodiments, an antibody of the invention is modified using coupling techniques known in the art, including, but not limited to, enzymatic means, oxidative substitution, and chelation. Modifications can be used, for example, for attachment of labels for immunoassay. Modified polypeptides are made using established procedures in the art and can be screened using standard assays known in the art, some of which are described below.

The antibody or polypeptide of the invention may be conjugated (for example, linked) to an agent, such as a therapeutic agent or a label. Examples of therapeutic agents are radioactive moieties, cytotoxins, and chemotherapeutic molecules.

The antibody (or polypeptide) of this invention may be linked to a label such as a fluorescent molecule, a radioactive molecule, an enzyme, or any other labels known in the art. As used herein, the term “label” refers to any molecule that can be detected. In a certain embodiment, an antibody may be labeled by incorporation of a radiolabeled amino acid. In a certain embodiment, biotin moieties that can be detected by marked avidin (e.g., streptavidin containing a fluorescent marker or enzymatic activity that can be detected by optical or colorimetric methods) may be attached to the antibody. In certain embodiments, a label may be incorporated into or attached to another reagent which in turn binds to the antibody of interest. For example, a label may be incorporated into or attached to an antibody that in turn specifically binds the antibody of interest. In certain embodiments, the label or marker can also be therapeutic. Various methods of labeling polypeptides and glycoproteins are known in the art and may be used. Certain general classes of labels include, but are not limited to, enzymatic, fluorescent, chemiluminescent, and radioactive labels. Examples of labels for polypeptides include, but are not limited to, the following: radioisotopes or radionucleotides (e.g., ³H, ¹⁴C, ¹⁵N, ³⁵S, ⁹⁰Y, ⁹⁹Tc, ¹¹¹In, ¹²⁵I, or ¹³¹I), fluorescent labels (e.g., fluorescein isothocyanate (FITC), rhodamine, lanthanide phosphors, or phycoerythrin (PE)), enzymatic labels (e.g., horseradish peroxidase, β-galactosidase, luciferase, alkaline phosphatase, glucose oxidase, glucose-6-phosphate dehydrogenase, alcohol dehydrogenase, malate dehydrogenase, penicillinase, or luciferase), chemiluminescent, biotinyl groups, predetermined polypeptide epitopes recognized by a secondary reporter (e.g., leucine zipper pair sequences, binding sites for secondary antibodies, metal binding domains, or epitope tags). In certain embodiments, labels are attached by spacer arms of various lengths to reduce potential steric hindrance.

The invention also provides pharmaceutical compositions comprising antibodies or polypeptides described herein, and a pharmaceutically acceptable carrier or excipients. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

In some embodiments, the invention provides compositions (described herein) for use in any of the methods described herein, whether in the context of use as a medicament and/or use for manufacture of a medicament.

### Polynucleotides, Vectors and Host Cells

The invention also provides polynucleotides comprising a nucleotide sequence encoding any of the antibodies and polypeptides described herein. In some embodiments, the polypeptides comprise the sequences of light chain and heavy chain variable regions.

In some embodiments, the polynucleotides comprise a nucleic acid sequence encoding the heavy chain variable region of antibody 5F4, and/or a nucleic acid sequence encoding the light chain variable region of antibody 5F4 (such as a nucleic acid encoding the amino acid sequence in FIG. 2A or 2B or a fragment thereof). In some embodiments, the polynucleotides comprise a nucleic acid sequence encoding a heavy chain variable region comprising one, two, or three CDRs of antibody 5F4, and/or a nucleic acid sequence encoding a light chain variable region comprising one, two, or three CDRs of antibody 5F4 (such as a CDR from the sequence in FIG. 2A or 2B). In some embodiments, the polynucleotides comprise a nucleic acid sequence of antibody 5F4. In some embodiments, the nucleic acid consists of or comprises SEQ ID NO: 2 and/or 4.

In some embodiments, the polynucleotides comprise a nucleic acid sequence encoding the heavy chain variable region of antibody 5F4Ac.2/v17, and/or a nucleic acid sequence encoding the light chain variable region of antibody 5F4Ac.2/v17 (such as a nucleic acid encoding the amino acid sequence of SEQ ID NO:19 and/or 20 or a fragment thereof). In some embodiments, the polynucleotides comprise a nucleic acid sequence encoding a heavy chain variable region comprising one, two, or three CDRs of antibody 5F4Ac.2/v17, and/or a nucleic acid sequence encoding a light chain variable region comprising one, two, or three CDRs of antibody 5F4Ac.2/v17 (such as a CDR from the sequence in SEQ ID NO:19 and/or 20).

In some embodiments, the polynucleotides comprise a nucleic acid sequence encoding the heavy chain variable region of antibody 3B6.1, and/or a nucleic acid sequence encoding the light chain variable region of antibody 3B6.1 (such as a nucleic acid encoding the amino acid sequence in FIG. 3A or 3B or a fragment thereof). In some embodiments, the polynucleotides comprise a nucleic acid sequence encoding a heavy chain variable region comprising one, two, or three CDRs of antibody 3B6.1, and/or a nucleic acid sequence encoding a light chain variable region comprising one, two, or three CDRs of antibody 3B6.1 (such as a CDR from the sequence in FIG. 3A or 3B). In some embodiments, the polynucleotides comprise a nucleic acid sequence of antibody 3B6.1. In some embodiments, the nucleic acid consists of or comprises SEQ ID NO: 6 and/or 8.

In some embodiments, the polynucleotides comprise a nucleic acid sequence encoding the heavy chain variable region of antibody 6A4.28, and/or a nucleic acid sequence encoding the light chain variable region of antibody 6A4.28 (such as a nucleic acid encoding the amino acid sequence in FIG. 4A or 4B or a fragment thereof). In some embodiments, the polynucleotides comprise a nucleic acid sequence encoding a heavy chain variable region comprising one, two, or three CDRs of antibody 6A4.28, and/or a nucleic acid sequence encoding a light chain variable region comprising one, two, or three CDRs of antibody 6A4.28 (such as a CDR from the sequence in FIG. 4A or 4B). In some embodiments, the polynucleotides comprise a nucleic acid sequence of antibody 6A4.28. In some embodiments, the nucleic acid consists of or comprises SEQ ID NO: 10 and/or 12.

In some embodiments, the polynucleotides comprise a nucleic acid sequence encoding the heavy chain variable region of antibody 9A6.2, and/or a nucleic acid sequence encoding the light chain variable region of antibody 9A6.2 (such as a nucleic acid encoding the amino acid sequence in FIG. 5A or 5B or a fragment thereof). In some embodiments, the polynucleotides comprise a nucleic acid sequence encoding a heavy chain variable region comprising one, two, or three CDRs of antibody 9A6.2, and/or a nucleic acid sequence encoding a light chain variable region comprising one, two, or three CDRs of antibody 9A6.2 (such as a CDR from the sequence in FIG. 5A or 5B). In some embodiments, the polynucleotides comprise a nucleic acid sequence of antibody 9A6.2. In some embodiments, the nucleic acid consists of or comprises SEQ ID NO: 14 and/or 16.

It is appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Thus, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions, and/or substitutions of nucleotides. The resulting mRNA and protein can, but need not, have an altered structure or function. Alleles can be identified using standard techniques (such as hybridization, amplification, and/or database sequence comparison).

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides can be inserted into host cells by any means known in the art. Cells are transformed by introducing an exogenous polynucleotide by direct uptake, endocytosis, transfection, F-mating, or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al. (1989).

Alternatively, PCR allows reproduction of DNA sequences. PCR technology is well known in the art and is described in U.S. Pat. Nos. 4,683,195; 4,800,159; 4,754,065; and 4,683,202, as well as PCR: The Polymerase Chain Reaction, Mullis et al. eds., Birkauswer Press, Boston (1994).

The invention also provides vectors (e.g., cloning vectors or expression vectors) comprising a nucleic acid sequence encoding any of the polypeptides (including antibodies) described herein. Suitable cloning vectors can be constructed according to standard techniques or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the invention. The expression vector may replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; and suitable transcriptional controlling elements (such as promoters, enhancers, or terminator). For expression (i.e., translation), one or more translational controlling elements are also usually required, such as ribosome binding sites, translation initiation sites, or stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides often depends on features of the host cell.

The invention also provides host cells comprising any of the polynucleotides or vectors described herein. Any host cells capable of over-expressing heterologous DNAs can be used for the purpose of isolating the genes encoding the antibody, polypeptide, or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe, or K. lactis).

### Diagnostic Uses

The present invention provides methods of using the antibodies, polypeptides, and polynucleotides of the present invention for detection, diagnosis, monitoring, and therapy selection for plasmacytoma, multiple myeloma, colorectal, gastric, or esophageal cancer associated with ORP150 polypeptide expression or expression of an epitope in an extracellular domain of an ORP150 polypeptide (either increased or decreased relative to a normal sample, and/or inappropriate expression, such as presence of expression in tissues(s) and/or cell(s) that normally lack ORP150 polypeptide expression).

In some embodiments, the method comprises detecting ORP150 polypeptide expression (or expression of an epitope in an extracellular domain of an ORP150 polypeptide) in a sample obtained from a subject suspected of having plasmacytoma, multiple myeloma, colorectal, gastric, and/or esophageal cancer. In some embodiments, the method of detection comprises contacting the sample with an antibody, polypeptide, or polynucleotide of the present invention and determining whether the level of binding differs from that of a control or comparison sample (e.g., the level of binding to the cell surface). The method is useful to determine whether the subject has plasmacytoma, multiple myeloma, colorectal, gastric, and/or esophageal cancer or has an increased risk for plasmacytoma, multiple myeloma, colorectal, gastric, and/or esophageal cancer. The method is also useful to determine whether the antibodies or polypeptides described herein are an appropriate treatment for the subject. In some embodiments, the methods further comprise treating a subject diagnosed or selected for treatment by administering an effective amount of one or more antibodies or polypeptides described herein.

The present invention provides methods for diagnosing plasmacytoma or multiple myeloma or an increased risk for plasmacytoma or multiple myeloma in an individual comprising contacting a bone marrow sample from the individual with of one or more antibodies or polypeptides described herein that bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of a bone marrow cell indicates that the individual has plasmacytoma or multiple myeloma or has an increased risk for plasmacytoma or multiple myeloma.

The present invention provides methods for diagnosing colorectal cancer or an increased risk for colorectal cancer in an individual comprising contacting a sample comprising a colon or rectum cell (such as a biopsy or other surgical sample) from the individual with of one or more antibodies or polypeptides described herein that bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of the colon or rectum cell indicates that the individual has colorectal cancer or has an increased risk for colorectal cancer.

The present invention provides methods for diagnosing gastric cancer or an increased risk for gastric cancer in an individual comprising contacting a sample comprising a gastric cell (such as a biopsy or other surgical sample) from the individual with of one or more antibodies or polypeptides described herein that bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of the gastric cell indicates that the individual has gastric cancer or has an increased risk for gastric cancer.

The present invention provides methods for diagnosing esophageal cancer or an increased risk for esophageal cancer in an individual comprising contacting a sample comprising an esophageal cell (such as a biopsy or other surgical sample) from the individual with of one or more antibodies or polypeptides described herein that bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of the esophageal cell indicates that the individual has esophageal cancer or has an increased risk for esophageal cancer.

The present invention provides methods for selecting a therapy for an individual having plasmacytoma or multiple myeloma comprising contacting a bone marrow sample from the individual with of one or more antibodies or polypeptides described herein the bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of a bone marrow cell indicates that the one or more antibodies or polypeptides described herein are useful for treating the plasmacytoma or multiple myeloma in the individual.

The present invention provides methods for selecting a therapy for an individual having colorectal cancer in an individual comprising contacting a sample comprising a colon or rectum cell (such as a biopsy or other surgical sample) from the individual with of one or more antibodies or polypeptides described herein that bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of the colon or rectum cell indicates that the one or more antibodies or polypeptides described herein are useful for treating the colorectal cancer in the individual.

The present invention provides methods for selecting a therapy for an individual having gastric cancer in an individual comprising contacting a sample comprising a gastric cell (such as a biopsy or other surgical sample) from the individual with of one or more antibodies or polypeptides described herein that bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of the gastric cell indicates that the one or more antibodies or polypeptides described herein are useful for treating the gastric cancer in the individual.

The present invention provides methods for selecting a therapy for an individual having esophageal cancer in an individual comprising contacting a sample comprising an esophageal cell (such as a biopsy or other surgical sample) from the individual with of one or more antibodies or polypeptides described herein that bind to an ORP150 polypeptide expressed on the cell surface of a plasmacytoma cell, multiple myeloma cell, colorectal cancer cell, gastric cancer cell, or esophageal cancer cell, whereby the ability of one or more of the antibodies or polypeptides to bind to the cell surface of the esophageal cell indicates that the one or more antibodies or polypeptides described herein are useful for treating the esophageal cancer in the individual.

As used herein, the term “a sample” or “a biological sample” refers to a whole organism or a subset of its tissues, cells, or component parts (e.g., body fluids, including, but not limited to, blood, plasma, serum, bone marrow, and urine). “A sample” or “a biological sample” further refers to a homogenate, lysate, or extract prepared from a whole organism or a subset of its tissues, cells, or component parts, or a fraction or portion thereof, including, but not limited to, for example, blood cells, bone marrow, biopsy or other surgical sample (such as biopsies of the colon and/or rectum), external sections of the skin, respiratory, intestinal, and genitourinary tracts, tumors, and organs. Most often, the sample has been removed from an animal, but the term “a sample” or “a biological sample” can also refer to cells or tissue analyzed in vivo, i.e., without removal from animal. Typically, “a sample” or “a biological sample” contains cells from the animal, but the term can also refer to non-cellular biological material, such as non-cellular fractions of blood, saliva, or urine that can be used to measure the cancer-associated polynucleotide or polypeptides levels. In some embodiments, a sample is at least partially purified before use. For example, one or more cell types of interest (such as plasma cells) can be removed from a sample (such as a bone marrow sample) for analysis of the binding of an antibody or polypeptide of the invention to the one or more cell types of interest. “A sample” or “a biological sample” further refers to a medium, such as a nutrient broth or gel in which an organism has been propagated, which contains cellular components, such as proteins or nucleic acid molecules.

In one embodiment, the cells or cell/tissue lysate are contacted with an antibody or polypeptide and the binding between the antibody (or polypeptide) and the cell is determined. When the test cells are shown binding activity as compared to a control cell of the same tissue type, it may indicate that the test cell is cancerous. In some embodiments, the test cells are from human tissues.

Various methods known in the art for detecting specific antibody-antigen binding can be used. Immunohistochemistry methods using the anti-ORP150 antibodies described herein may be used to detect the presence and/or quantity of the ORP150 protein on the cell surface in a sample. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. Appropriate labels include, without limitation, radionuclides (e.g., ¹²⁵I, ¹³¹I, ³⁵S, ³H, or ³²P), enzymes (e.g., alkaline phosphatase, horseradish peroxidase, luciferase, or β-galactosidase), fluorescent moieties or proteins (e.g., fluorescein, rhodamine, phycoerythrin, GFP, or BFP), or luminescent moieties (e.g., Qdot™ nanoparticles supplied by the Quantum Dot Corporation, Palo Alto, Calif.). General techniques to be used in performing the various immunoassays noted above are known to those of ordinary skill in the art.

For purposes of diagnosis, the polypeptide including antibodies can be labeled with a detectable moiety including, but not limited to, radioisotopes, fluorescent labels, and various enzyme-substrate labels know in the art. Methods of conjugating labels to an antibody are known in the art.

In some embodiments, the polypeptides including antibodies of the invention need not be labeled, and the presence thereof can be detected using a labeled antibody which binds to the antibodies of the invention.

The antibodies of the present invention can be employed in any known assay method, such as competitive binding assays, direct and indirect sandwich assays, and immunoprecipitation assays. Zola, Monoclonal Antibodies: A Manual of Techniques, pp. 147-158 (CRC Press, Inc. 1987).

The antibodies and polypeptides can also be used for in vivo diagnostic assays, such as in vivo imaging. Generally, the antibody or the polypeptide is labeled with a radionuclide (such as ¹¹¹In, ⁹⁹Tc, ¹⁴C, ¹³¹I, ¹²⁵I, or ³H) so that the cells or tissue of interest can be localized using immunoscintiography.

The antibody may also be used as staining reagent in pathology using techniques well known in the art.

### Therapeutic and Prophylactic Uses

A striking feature of the antibodies of the present invention relates to their ability to effectively induce plasmacytoma or gastric cancer cell death (such as cell death by apoptosis). While not intending to be bound by any particular mechanism, since the antibodies also bind plasmacytoma, multiple myeloma, colorectal, gastric, and esophageal cancer cells, they may be used to induce complement-dependent cytotoxicity and/or antibody-dependent cell-mediated cytotoxicity in plasmacytoma, multiple myeloma, colorectal, gastric, or esophageal cancer cells after binding to the cell surface of the cell. Alternatively or additionally, the antibodies may be linked to a cytotoxic agent to kill or inhibit the growth of plasmacytoma, multiple myeloma, colorectal, gastric, or esophageal cancer cells.

Thus, the present invention provides therapeutic and prophylactic uses of the antibodies and polypeptides of the present invention (or polynucleotides encoding them) in treating, preventing, or delaying the development of plasmacytoma, multiple myeloma, colorectal cancer, gastric cancer, and/or esophageal cancer. The method may further comprise a step of detecting the binding between an antibody or a polypeptide described herein and a tumor or cancer cell in an individual to be treated.

Generally, an effective amount of a composition comprising an antibody or a polypeptide is administered to a subject in need of treatment (such as an individual having plasmacytoma, multiple myeloma, colorectal cancer, gastric cancer and/or esophageal cancer, or an individual having an increased risk for plasmacytoma, multiple myeloma, colorectal cancer, gastric cancer and/or esophageal cancer), thereby inhibiting, delaying, or preventing growth of the cancer cell (such as a plasmacytoma cell, multiple myeloma cell, colorectal cancer cell, gastric cancer cell, or esophageal cancer cell) and/or inducing death of the cancer cell. Desirably, the composition is formulated with a pharmaceutically acceptable carrier.

Additionally, the present invention provides therapeutic and prophylactic uses of anti-idiotypic antibodies (or polypeptides derived from them or polynucleotides encoding them) that specifically bind to a primary antibody that recognizes an ORP150 polypeptide expressed on the cell surface of a plasmacytoma cell, multiple myeloma cell, colorectal cancer cell, gastric cancer cell, or esophageal cancer cell. Generally, an effective amount of a composition comprising an anti-idiotypic antibody or a polypeptide is administered to a subject in need of treatment (such as an individual having plasmacytoma, multiple myeloma, colorectal cancer, gastric cancer and/or esophageal cancer, or an individual having an increased risk for plasmacytoma, multiple myeloma, colorectal cancer, gastric cancer and/or esophageal cancer), thereby inhibiting, delaying, or preventing growth of the cancer cell (such as a plasmacytoma cell, multiple myeloma cell, colorectal cancer cell, gastric cancer cell, or esophageal cancer cell) and/or inducing death of the cancer cell. Desirably, the composition is formulated with a pharmaceutically acceptable carrier. In some embodiments, an extracellular domain or fragment thereof of an ORP150 polypeptide expressed on the cell surface of a plasmacytoma cell, multiple myeloma cell, colorectal cancer cell, gastric cancer cell, or esophageal cancer cell is also administered to the individual.

The present invention also features therapeutic and prophylactic uses of ORP150 polypeptides or fragments thereof, or a polypeptide comprising an ORP150 fragment (or polynucleotides encoding them) in treating, preventing, or delaying the development of plasmacytoma, multiple myeloma, colorectal cancer, gastric cancer and/or esophageal cancer. The method may further comprise a step of detecting the binding between an antibody or a polypeptide described herein and a tumor or cancer cell in an individual to be treated. In some embodiments, the polypeptide comprises amino acids 723-732, 673-752, 701-800, 673-800 of SEQ ID NO:17.

Generally, an effective amount of a composition (such as a vaccine composition) of a polypeptide comprising an ORP150 polypeptide or fragment thereof of obtained from or expressed on the cell surface of a plasmacytoma cell, multiple myeloma cell, colorectal cancer cell, gastric cancer cell, or esophageal cancer cell is administered to a subject in need of treatment (such as an individual having plasmacytoma, multiple myeloma, colorectal cancer, gastric cancer and/or esophageal cancer or an individual having an increased risk for plasmacytoma, multiple myeloma, colorectal cancer, gastric cancer and/or esophageal cancer). In some embodiments, one or more antibodies that bind to the cell surface of a plasmacytoma, multiple myeloma, colorectal cancer cell, gastric cancer cell, or esophageal cancer cell in the individual are generated by the individual. In some embodiments, the ORP150 polypeptide fragment is an extracellular domain or fragment thereof from an ORP150 polypeptide expressed on the cell surface of a plasmacytoma cell, multiple myeloma cell, colorectal cancer cell, gastric cancer cell, or esophageal cancer cell. In some embodiments, a polypeptide comprising amino acids 723-732, 673-752, or 701-800 of SEQ ID NO:17 is administered to a subject in need of treatment. Desirably, the composition is formulated with a pharmaceutically acceptable carrier.

In another embodiment, the present invention also contemplates administration of a composition comprising the antibodies or polypeptides of the present invention conjugated to other molecules, such as detectable labels, or therapeutic or cytotoxic agents. The agents may include, but are not limited to radioisotopes, toxins, toxoids, inflammatory agents, enzymes, antisense molecules, peptides, cytokines, and chemotherapeutic agents. Methods of conjugating the antibodies with such molecules are generally known to those of skilled in the art. See, e.g., PCT publications WO 92/08495; WO 91/14438; WO 89/12624; U.S. Pat. No. 5,314,995; and EP 396,387; the disclosures of which are incorporated herein by reference in their entireties.

In one embodiment, the composition comprises an antibody or polypeptide conjugated to a cytotoxic agent. Cytotoxic agents can include any agents that are detrimental to cells. An exemplary class of cytotoxic agents that can be conjugated to the antibodies or fragments may include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and analogs or homologs thereof.

In one embodiment, any of the compositions described herein is formulated for administration by intraperitoneal, intravenous, subcutaneous, or intramuscular injections, or other forms of administration such as oral, mucosal, via inhalation, sublingually, etc.

The dosage required for the treatment depends on the choice of the route of administration, the nature of the formulation, the nature of the subject's illness, the subject's size, weight, surface area, age and sex; other drugs being administered, and the judgment of the attending physician. Suitable dosages are in the range of 0.01-1000.0 mg/kg.

Generally, any of the following doses may be used: a dose of greater than or about 50 mg/kg body weight; greater than or about 10 mg/kg body weight; greater than or about 3 mg/kg body weight; greater than or about 1 mg/kg body weight; greater than or about 750 μg/kg body weight; greater than or about 500 μg/kg body weight; greater than or about 250 μg/kg body weight; greater than or about 100 μg/kg body weight; greater than or about 50 μg/kg body weight; greater than or about 10 μg/kg body weight; greater than or about 1 μg/kg body weight, or less, is administered. For repeated administrations over several days or longer, depending on the condition, the treatment is sustained until a desired suppression of disease symptoms occurs. An exemplary dosing regimen comprises administering a weekly dose of about 6 mg/kg of the antibody or polypeptide. However, other dosage regimens may be useful, depending on the pattern of pharmacokinetic decay that the practitioner wishes to achieve. Empirical considerations, such as the half-life, generally will contribute to determination of the dosage. The progress of this therapy is easily monitored by conventional techniques and assays.

In some subjects, more than one dose may be required. Frequency of administration may be determined and adjusted over the course of therapy. For example, frequency of administration may be determined or adjusted based on the type and stage of the cancer to be treated, whether the agent is administered for preventive or therapeutic purposes, previous therapy, the patient's clinical history and response to the agent, and the discretion of the attending physician. Typically the clinician will administer a therapeutic antibody (such as humanized 5F4), until a proper dosage is reached to achieves the desired result. In some cases, sustained continuous release formulations of antibodies may be appropriate. Various formulations and devices for achieving sustained release are known in the art.

In one embodiment, dosages for the antibodies or polypeptides may be determined empirically in subjects who have been given one or more administration(s). Subjects are given incremental dosages of the antibodies or polypeptides. To assess efficacy of the antibodies or polypeptides, markers of the disease symptoms such as an ORP150 polypeptide can be monitored. Efficacy in vivo can also be measured by assessing tumor burden or volume, the time to disease progression (TDP), and/or determining the response rates (RR).

Administration of an antibody or polypeptide in accordance with the method in the present invention can be continuous or intermittent, depending, for example, upon the recipient's physiological condition, whether the purpose of the administration is therapeutic or prophylactic, and other factors known to skilled practitioners. The administration of an antibody or a polypeptide may be essentially continuous over a preselected period of time or may be in a series of spaced dose.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles, and unilamellar vesicles.

In another embodiment, the composition can comprise one or more anti-cancer agents, one or more antibodies described herein, or with an antibody or polypeptide that binds to a different antigen. Such composition can contain at least one, at least two, at least three, at least four, or at least five different antibodies. The antibodies and other anti-cancer agents may be in the same formulation (e.g., in a mixture, as they are often denoted in the art), or in separate formulations but are administered concurrently or sequentially, are particularly useful in treating a broader range of population of individuals.

A polynucleotide encoding any of the antibodies (such as antibody 5F4 or a humanized form) or polypeptides (such as a polypeptide comprising an ORP150 polypeptide or fragment thereof) of the present invention can also be used for delivery and expression of any of the antibodies or polypeptides of the present invention in a desired cell. It is apparent that an expression vector can be used to direct expression of the antibody or polypeptide. The expression vector can be administered by any means known in the art, such as intraperitoneally, intravenously, intramuscularly, subcutaneously, intrathecally, intraventricularly, orally, enterally, parenterally, intranasally, dermally, sublingually, or by inhalation. For example, administration of expression vectors includes local or systemic administration, including injection, oral administration, particle gun or catheterized administration, and topical administration. One skilled in the art is familiar with administration of expression vectors to obtain expression of an exogenous protein in vivo. See, e.g., U.S. Pat. Nos. 6,436,908; 6,413,942; and 6,376,471.

Targeted delivery of therapeutic compositions comprising a polynucleotide encoding any of the antibodies or polypeptides of the present invention can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al. (1990), Proc. Natl. Acad. Sci. USA, 87:3655; Wu et al. (1991), J. Biol. Chem. 266:338. Therapeutic compositions containing a polynucleotide are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol.

The therapeutic polynucleotides and polypeptides of the present invention can be delivered using gene delivery vehicles. The gene delivery vehicle can be of viral or non-viral origin (see generally, Jolly (1994), Cancer Gene Therapy 1:51; Kimura (1994), Human Gene Therapy 5:845; Connelly (1985), Human Gene Therapy 1:185; and Kaplitt (1994), Nature Genetics 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses, e.g., PCT Publication Nos. WO 90/07936; WO 94/03622; WO 93/25698; WO 93/25234; WO 93/11230; WO 93/10218; WO 91/02805; U.S. Pat. Nos. 5,219,740; 4,777,127; GB Patent No. 2,200,651; and EP Patent No. 0 345 242; alphavirus-based vectors, e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors, e.g., PCT Publication Nos. WO 94/12649, WO 93/03769; WO 93/19191; WO 94/28938; WO 95/11984; and WO 95/00655. Administration of DNA linked to killed adenovirus as described in Curiel (1992), Hum. Gene Ther. 3:147 can also be employed.

Non-viral delivery vehicles and methods can also be employed, including, but are not limited to, polycationic condensed DNA linked or unlinked to killed adenovirus alone (see, e.g., Curiel (1992), Hum. Gene Ther. 3:147); ligand-linked DNA (see, e.g., Wu (1989), J. Biol. Chem. 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes.

Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent NO. 0 524 968. Additional approaches are described in Philip (1994), Mol. Cell Biol. 14:2411 and in Woffendin (1994), Proc. Natl. Acad. Sci. 91:1581.

The composition comprising an antibody or polypeptide of the present invention can be administered sequentially or concurrently with one or more other therapeutic agents such as chemotherapeutic agents (such as 5-FU, 5-FU/MTX, 5-FU/Leucovorin, Levamisole, Irinotecan, Oxaliplatin, Capecitabin, or Uracil/Tegafur), immunoadjuvants, growth inhibitory agents, cytotoxic agents, cytokines, etc. The amounts of the antibody (or polypeptide) and the therapeutic agent depend on what type of drugs are used, the pathological condition being treated, and the scheduling and routes of administration but would generally be less than if each were used individually.

Following administration of the composition comprising the antibody or polypeptide described herein, the efficacy of the composition can be evaluated both in vitro and in vivo by various methods well known to one of ordinary skill in the art. Various animal models are well known for testing anti-cancer activity of a candidate composition. These include human tumor xenografting into athymic nude mice or scid/scid mice, or genetic murine tumor models such as p53 knockout mice. The in vivo nature of these animal models make them particularly predictive of responses in human patients. Such models can be generated by introducing cells into syngeneic mice using standard techniques, e.g., subcutaneous injection, tail vein injection, spleen implantation, intraperitoneal implantation, implantation under the renal capsule, etc.

### Kits

The invention also provides kits comprising an antibody or a polypeptide described herein for use in the instant methods. Kits of the invention may include one or more containers comprising a purified antibody or a polypeptide described herein. The kits may further comprise instructions for use in accordance with any of the methods of the invention described herein. In some embodiments, these instructions comprise a description of administration of the antibody to treat, prevent, or delay development of plasmacytoma, multiple myeloma, colorectal cancer, gastric cancer, or esophageal cancer, according to any of the methods described herein. The kit may further comprise a description of selecting an individual suitable for treatment based on identifying whether that individual has the disease and the stage of the disease, or whether an ORP150 polypeptide or an epitope in an ORP150 polypeptide (such as an epitope in an extracellular domain of an ORP150 polypeptide) is expressed on the cancer cells in the individual.

In some embodiments, the kits for detecting a cancer cell in a sample comprise an antibody or a polypeptide described herein and one or more reagents for detecting binding of the antibody or the polypeptide to a cell in the sample.

The instructions relating to the use of the antibodies or polypeptides to treat, prevent, or delay development of plasmacytoma, multiple myeloma, colorectal cancer, gastric cancer, or esophageal cancer generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, preventing, or delaying development of a cancer described herein. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer), or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an antibody or polypeptide described herein. The container may further comprise a second pharmaceutically active agent.

Kits may optionally provide additional components such as buffers and interpretive information. Normally, the kit comprises a container and a label or package insert(s) on or associated with the container.

### EXAMPLES

The following Examples are provided to illustrate but not to limit the invention. It is noted that similar methods may be performed on multiple myeloma, colorectal, gastric, or esophageal cancer cells (such as cells isolated from human multiple myeloma, colorectal cancer, gastric cancer, or esophageal cancer patients) to measure the ability of an antibody to bind, induce apoptosis, induce antibody-dependent cell-mediated cytotoxicity and/or induce complement-dependent cytotoxicity of multiple myeloma, colorectal, gastric, or esophageal cancer cells.

### Example 1

### Generation and Characterization of Antibodies that Specifically Bind Plasmacytoma Cells, Colorectal, Gastric, and Esophageal Cancer Cells

### Generation of Monoclonal Antibodies

A female 8-week BALB/c mouse was immunized with the RPMI8226 (ATCC# CCL-155) cell membrane, and spleen cells were finally fused with P3X63 myeloma cells. Hybridomas were selected with DMEM supplemented with 10% FBS (Hyclone) and containing HAT (Hybri-Max®, Sigma H0262, at a final concentration of 100 μM hypoxanthine, 0.4 μM aminopterin, and 16 μM thymidine). RPMI8226 cells were grown in RPMI 1640 medium (GIBCO BRL) with 10% FBS (Hyclone), 100 units/ml of penicillin, and 100 μg/ml of streptomycin (GIBCO BRL) at 37° C. in a humidified atmosphere of 5% CO₂. Resulting hybridomas were screened for their ability to induce apoptosis of RPMI8226 cells (10% higher than background). One monoclonal antibody of the IgM subclass was identified: antibody 5F4. The sequences of the variable region of the heavy and light chain of antibody 5F4 (IgM, K) are shown in FIGS. 2A and 2B. Constant region sequences of mouse IgM and Kappa light chain isotype are described in Kehry et al., Proc. Natl. Acad. Sci. U.S.A. 76:2932-2936, 1979; and Kawakami et al., Nucleic Acids Res. 17:3933-3945, 1980; and Hamlyn et al., Nucleic Acids Res. 18:4485-4494, 1981.

### Monoclonal Antibody 5F4 Specifically Bind Plasmacytoma Cells, Colorectal Cancer Cells, Gastric Cancer Cells, and Esophageal Cancer Cells

Monoclonal antibody 5F4 was shown to bind positive to human plasmacytoma cell lines (RPMI8226 (ATCC# CCL-155), U266 (ATCC# TIB-196), NCI-H929 (ATCC# CRL-9068), and L363 cells (DSMZ# ACC 49, Germany)) and colorectal cancer cells (Colo205 (ATCC# CCL-222), DLD-1 (ATCC# CCL-221), and HT29 (ATCC# HTB-38)), gastric cancer cells (SNU-1 (ATCC# CRL-5971), Kato-III (ATCC# HTB-103), and esophageal cancer cells (CE146T (BCRC# 60617, Taiwan) by flow cytometry.

### Monoclonal Antibody 5F4 Induces Apoptosis of Human Plasmacytoma Cells

Monoclonal antibody 5F4 was tested for its ability to induce apoptosis in various cancer cell lines in vitro. Yo-Pro1 stains nucleic acid under the circumstances of degradation of nuclear envelope, which is characteristic of early apoptotic cells. Thus, staining of Yo-Pro1, as detected by FACS analysis, was used to measure apoptosis induced by antibody 5F4. The cells were incubated in the presence of purified antibodies (at concentrations indicated) in a 96-well plate at 37° C. for 6 hours. At the end of incubation, cells were stained with Yo-Pro1 (Molecular Probes) and analyzed by flow cytometry. The results (Table 1) showed that antibody 5F4 was able to induce apoptosis of the human plasmacytoma cell lines. On contrast, no apoptotic effect was detected on the colorectal, gastric, or esophageal cancer cell lines tested.

TABLE 1
Apoptosis of Human Plasmacytoma Cells¹

| | Cell Line | | | | | | | | | | | |
|---|---|---|---|---|---|---|---|---|---|---|---|---|
| | RPMI 8226 | | | U266 | | | NCI-H929 | | | L363 | | |
| Antibody | 3 μg/ml | 1 μg/ml | 0.3 μg/ml | 3 μg/ml | 1 μg/ml | 0.3 μg/ml | 3 μg/ml | 1 μg/ml | 0.3 μg/ml | 10 μg/ml | 3 μg/ml | 1 μg/ml |
| 5F4 | 34 | 34 | 28 | 32 | 33 | 24 | 17 | 19 | 16 | 37 | 43 | 50 |
| Untreated | 21 | | | 7 | | | 11 | | | 12 | | |

¹Values shown are percent Yo-Pro1 positive cells.

### Monoclonal Antibody 5F4 Induces Complement-Dependent Cytotoxicity of Human Plasmacytoma Cells

Monoclonal antibody 5F4 was also tested for its ability to induce complement-dependent cytotoxicity (CDC) in the three human plasmacytoma cell lines. Human serum used in the CDC assay was prepared from healthy volunteers after clotting. Briefly, 2×10⁵ cells (RPMI8226, U266, and NCI-H929 cells) per well were first incubated with the antibody (at concentrations indicated) at 4° C. for 30 minutes, and then incubated with 20% human serum at 37° C. for additional 30 minutes. At the end of the incubation, cells were stained with propidium iodide (PI; Sigma), which indicated lytic cells, and analyzed using a flow cytometer. Table 2 showed the percentage of PI positive (dead) cells when incubated with 1.1 to 30 ug/ml of 5F4 and also control antibody (background) in the CDC activity. At the concentration of 30 ug/ml, the CDC effect of 5F4 is 21.1%, 14.6% and 11.9% above background for H929, U266, and RPMI8226 cells, respectively.

TABLE 2
CDC effect on human plasmacytoma cells¹

| | 30 ug/ml | 10 ug/ml | 3.3 ug/ml | 1.1 ug/ml | control |
|---|---|---|---|---|---|
| H929 | 30.9 | 23.9 | 11.8 | 9.2 | 9.8 |
| U266 | 29.9 | 33.2 | 20.1 | 13.2 | 15.3 |
| RPMI8226 | 25.0 | 27.0 | 18.8 | 13.2 | 13.1 |

¹Values shown are percent PI positive cells.

### Monoclonal Antibody 5F4 Specifically Binds Human ORP150 Polypeptide

Membrane proteins were immunoprecipitated from RPMI8226 cells as follows. Membrane proteins from RPMI8226 cells were isolated with extraction buffer (20 mM Tris-HCl, pH 7.4, 160 mM NaCl, and 1% CHAPS) containing protease inhibitors (Complete tabs; Roche Molecular Biochemicals). Membrane protein lysates were first pre-cleaned by incubating them with non-immune mouse immunoglobulin immobilized on Protein G beads (Amersham Pharmacia Biotech Inc., NJ) at 25° C. for 2 hours. The supernatant portion was directly applied to antibody 5F4 or to anti-human ORP150 antibody (B01, mouse polyclonal Abs; Novus) coupled Protein G beads for overnight incubation at 4° C. After extensive washing, the immunoprecipitated protein was eluted, mixed with an equal volume of sample buffer (50 mM Tris-HCl, pH 6.8, 100 mM DTT, 2% SDS, 0.1% bromophenol blue, 10% glycerol, and 2-ME), separated by 6% SDS-PAGE, and then transferred to a nitrocellulose membrane (Hybond-C Super, Amersham). The nitrocellulose membrane was then blocked with 5% skimmed milk in PBS, and incubated with antibody 5F4 (2 μg/ml) at room temperature for 1 hour. The blot was then treated with horseradish peroxidase-conjugated goat anti-mouse immunoglobulin (Jackson ImmunoResearch Laboratories, West Grove, Pa.) and developed with chemiluminescence reagents (ECL, Millipore Corp.) according to the manufacturer's instructions.

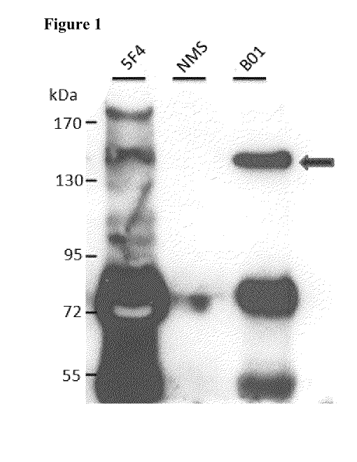

As shown in FIG. 1, ORP150 from the RPMI8226 membrane preparation was immunoprecipitated using mouse polyclonal anti-ORP150 antibody (B01) or antibody 5F4, and subsequently blotted with antibody 5F4 (MW ˜160 kD). The band was not shown in the control normal mouse serum (NMS) immunoprecipitated product. Therefore, antibody 5F4 recognizes human ORP150.

Monoclonal Antibody 5F4 does not Bind to Human Peripheral Blood Cells

Monoclonal antibody 5F4 was tested for its ability to bind to peripheral blood cells. Briefly, antibodies at optimal concentrations were added to cell suspensions and incubated for 30 minutes at 4° C. FITC- or PE-conjugated anti-mouse Ig (PharMingen) was used as a secondary antibody for the primary antibody 5F4. In some cases, cells were co-stained with antibody against certain surface markers (e.g., CD3, CD20, and CD14), and fluorescence intensity stained by antibody 5F4 was assessed in the gated population. Antibody 5F4 was also shown not to react with human RBC and platelets under the conditions tested. All flow cytometric analyses were performed on a BD-LSR flow cytometer (Becton Dickinson).

TABLE 3
Binding of antibody 5F4 to human normal blood cells¹

| | 5F4 | | | | | | |
|---|---|---|---|---|---|---|---|
| | 10 ug/ml | 3 ug/ml | 1 ug/ml | 0.3 ug/ml | 0.1 ug/ml | 2nd | NMS² |
| CD3+ cells | | | | | | | |
| Donor A | 6 | 6 | 6 | 6 | 6 | 11 | 8 |
| Donor B | 10 | 12 | 10 | 11 | 10 | 12 | 9 |
| Donor C | 11 | 12 | 12 | 10 | 10 | 9 | 13 |
| CD20+ cells | | | | | | | |
| Donor A | 11 | 11 | 11 | 7 | 12 | 14 | 29 |
| Donor B | 14 | 16 | 12 | 15 | 12 | 13 | 28 |
| Donor C | 14 | 19 | 16 | 11 | 11 | 10 | 19 |
| CD14+ cells | | | | | | | |
| Donor A | 51 | 46 | 41 | 36 | 33 | 28 | 58 |
| Donor B | 31 | 31 | 26 | 25 | 24 | 34 | 45 |
| Donor C | 26 | 26 | 27 | 22 | 22 | 17 | 50 |
| PMN cells | | | | | | | |
| Donor A | 4 | 4 | 3 | 3 | 3 | 2 | 3 |
| Donor B | 6 | 5 | 6 | 5 | 5 | 6 | 13 |
| Donor C | 5 | 6 | 6 | 5 | 6 | 7 | 9 |
| RPMI8226 (I) | 160 | 325 | 270 | 110 | 51 | | |
| RPMI8226 (II) | 314 | 575 | 428 | 196 | 78 | | |

¹Values shown are mean fluorescence intensity.
²NMS: normal mouse serum.

### Epitope Mapping for 5F4

The complete cDNA encoding human ORP150 (hORP150, UniProtKB/Swiss-Prot entry: Q9Y4L1) was cloned from U266 cell line (ATCC Cat. No. TIB-196) by RT-PCR. The C-terminal of hORP150 was linked with 3× Flag tag and cloned into the expression vector pcDNA5/FRT via restriction enzyme sites EcoR I and Xho I. Overlapping PCR was applied to engineer the deletion mutants of human ORP150. Mutants with deletion of amino acids 33-112, 113-192, 193-272, 273-352, 353-432, 433-512, 513-592, 593-672, 673-752, 753-832, 833-912, or 913-999 in human ORP150 were generated in Flp-In™ CHO cells (Invitrogen, Cat. No. R758-07) by transient transfection with Lipofectamine™ 2000 (Invitrogen, Cat. No. 11668-019). The supernatant and cell lysate of the transfected cells were prepared 72 hours after the transfection for epitope mapping.

Binding of 5F4 to the ORP150 wild type or mutant proteins generated from the supernatant of the transfected cells were tested using ELISA assay by coating the surface with anti-FLAG antibody, allowing binding of the ORP150 proteins in the supernatant to the anti-FLAG antibody, and detecting binding of 5F4 to the ORP150 proteins.

Binding of 5F4 to the wild type or the mutant ORP150 proteins from the supernatant and cell lysates was also tested using Western blot assays by immunoprecipitation the ORP150 proteins from the supernatant or cell lysates using anti-FLAG antibody, and detecting binding of 5F4 to the ORP150 proteins on the Western blot. The anti-Flag M2 affinity gel (Sigma, Cat. No. A2220) was used to immuno-precipitate the recombinant human ORP150 from the supernatant and cell lysates of the transfected cells. The IP products were subjected to SDS-PAGE and then transferred onto nitrocellulose membrane for Western blotting. After blocking with 6% milk in TBS, the membranes were blotted with 5F4 or anti-Flag M2 (Stratagene, Cat. No. 200472-21), followed by blotting with goat anti-mouse IgM-HRP (Jackson ImmunoReaserch, Cat. No. 115-035-075). The blotting signal was detected with Immobilon Western-Chemiluminescent HRP Substrate kits (Millipore, Cat. No. WBKLS0500)

Based on the Western blotting with anti-FLAG antibody, all the deletion mutants of hORP150 were successfully expressed in Flp-In™ CHO Cells. The ELISA assay and Western blotting with 5F4 demonstrated that 5F4 did not bind to ORP150 if amino acids 673-752 were deleted in the protein, and other deletions had no significant impact on binding of 5F4 to the ORP150 protein. This indicates that 5F4 binds to an epitope within amino acids 673-752. To further characterize the epitope, additional mutant ORP150 proteins were generated. The mutant ORP150 proteins contain deletion of amino acids 673-682, 683-692, 693-702, 703-712, 713-722, 723-732, 733-742, or 743-752. Binding of 5F4 to the ORP150 mutant proteins produced in the supernatant was tested using the ELISA assay, and binding of 5F4 to the ORP150 mutant proteins produced in the supernatant and the cell lysates were tested using the Western blotting. Data in these experiments demonstrated that 5F4 did not bind to ORP150 if amino acids 723-732 were deleted in the protein, and other deletions had no significant impact on binding of 5F4 to the ORP150 protein. These experiments indicate that 5F4 recognizes human ORP150 recombinantly produced, and the epitope of 5F4 locates at amino acids 723-732 (⁷²³LQDLTLRDLE⁷³² (SEQ ID NO:18)) of human ORP150. In addition, antibody 5F4 binds to the synthetic peptide having amino acid sequence LQDLTLRDLE (SEQ ID NO:18) as shown by an ELISA assay.

### Example 2

### Generation and Characterization of Additional Antibodies to ORP-150

The recombinant hORP150 amino acids 701-800 of SEQ ID NO:17 was cloned into the expression vector, pET32-a(+) (Merck Biosciences, Darmstadt, Germany), with His tag for subsequent purification. Mice were immunized with these purified protein fragments following the standard immunization protocol. Hybridomas were later generated by screening their binding to U266 cells on FACS. Three IgM antibodies that bound to U266 cells were identified: 3B6.1, 6A4.28, and 9A6.2.

These antibodies were further tested for their ability to induce CDC activity using 1× or 5× diluted hybridoma supernatant in cancer cell lines. The assay for testing CDC activity is described in detail in Example 1. The results are shown in Table 4 below. As shown in Table 4, antibody 3B6.1 bound to multiple myeloma cell U266, gastric cancer cell KatoIII, and colorectal cancer cell Colo205 and induced cell death through the CDC activity; antibody 6A4.28 bound to esophageal cancer cell CD146T and induced cell death through the CDC activity; and antibody 9A6.2 bound to gastric cancer cell KatoIII and induced cell death through the CDC activity.

TABLE 4
Binding and CDC activity of antibody 3B6.1, 6A4.28, and 9A6.2
3B6.1
(1) U266 (Multiple myeloma)

| Binding (MFI) | 2^{nd} Ab | 1x sup | 5x diluted sup |
|---|---|---|---|
| | 6 | 2779 | — |

| CDC (% PI+) | untreated | 1x sup | 5x diluted sup | C′ only |
|---|---|---|---|---|
| | 7.6 | 77 | 72 | 6.4 |

(2) KatoIII (Gastric cancer)

| Binding (MFI) | 2^{nd} Ab | 1x sup | 5x diluted sup |
|---|---|---|---|
| | 3 | 1154 | 863 |

| CDC (% PI+) | untreated | 1x sup | 5x diluted sup | C′ only |
|---|---|---|---|---|
| | 8.4 | 81 | 68 | 9.4 |

(3) Colo205 (Colorectal cancer)

| Binding (MFI) | 2^{nd} Ab | 1x sup | 5x diluted sup |
|---|---|---|---|
| | 2 | 72 | 185 |

| CDC (% PI+) | untreated | 1x sup | 5x diluted sup | C′ only |
|---|---|---|---|---|
| | 1.2 | 11 | 10 | 0.2 |

6A4.28
CE146T/VGH (Esophageal cancer)

| Binding (MFI) | 2^{nd} Ab | 1x sup | 5x diluted sup |
|---|---|---|---|
| | 3 | 559 | 759 |

| CDC (% PI+) | untreated | 1x sup | 5x diluted sup | C′ only |
|---|---|---|---|---|
| | 13 | 33 | 25 | 14 |

9A6.2
KatoIII (Gastric cancer)

| Binding (MFI) | 2^{nd} Ab | 1x sup | 5x diluted sup |
|---|---|---|---|
| | 3 | 43 | 53 |

| CDC (% PI+) | untreated | 1x sup | 5x diluted sup | C′ only |
|---|---|---|---|---|
| | 8.4 | 63 | 25 | 9.4 |

C′: Only complement was added, and no tested antibody was added.

Western blot was performed to confirm the binding specificity of antibody 3B6.1, 6A4.28 and 9A6.2. Recombinant protein ORP150 amino acids 701-800 and an unrelated protein (zinc-binding alcohol dehydrogenase domain containing 2) were run on a SDS-PAGE, and transferred to a nitrocellulose membrane. Antibody 3B6.1, 6A4.28 and 9A6.2 only recognized ORP150 amino acids 701-800, but not the unrelated protein on the nitrocellulose membrane. The results indicate that antibody 3B6.1, 6A4.28 and 9A6.2 specifically recognize ORP150 amino acids 701-800. The amino acid sequences of the heavy and light chain variable region of antibody 3B6.1 (IgM, K), 6A4.28 (IgM, K) and 9A6.2 (IgM, K) are shown in FIGS. 3A and 3B, 4A and 4B, and 5A and 5B, respectively.

### Monoclonal Antibody 3B6.1 Induces Apoptosis of Human Gastric Cancer Cells

Results also showed that monoclonal antibody 3B6.1 was able to induce apoptosis in human gastric cancer cell line, SNU-1. Briefly, cells were incubated in the presence of 3B6.1 hybridoma supernatants for 18 hours. At the end of incubation, cells were stained with Yo-Pro1 (Molecular Probes) for detection of apoptosis. 3B6.1 can induce 27% of Yo-Prot positive staining, compared to the 10% caused by the exhausted culture supernatant of a control hybridoma. In similar experiments, this antibody did not induce apoptosis in plasmacytoma cell line U266 or L363.

### Example 3

### Generation and Characterization of Chimeric and Humanized Antibodies

### Cloning of the Variable Regions of Light and Heavy Chains of Murine 5F4, 3B6.1, 6A4.28, and 9A6.2

The cDNA for variable regions (V region) of mouse hybridomas 5F4, 3B6.1, 6A4.28, and 9A6.2 light and heavy chains were amplified by PCR, and subcloned into pCRII-TOPO (Invitrogen) for sequence determination. Nucleotide sequences were obtained from several independent clones and analyzed. The mature amino acid sequences of the light and heavy chain V regions of 5F4 (IgM, κ), 3B6.1 (IgM, κ), 6A4.28 (IgM, κ), and 9A6.2 (IgM, κ) and the Kabat CDRs were identified as shown in FIG. 2 to FIG. 5.

Construction of Chimeric Antibodies from Murine Antibody 5F4

To generate vectors for expressing chimeric antibodies, cDNAs encoding the V_{L} and V_{H} regions of 5F4 were amplified by PCR using primers to include the 5′ signal peptide sequence and the 3′ splice donor signal. AvrII was introduced for light chain variable region cloning, and NheI was used for heavy chain variable region cloning. The cloning vectors contained the light chain κ constant region and the human heavy chain constant regions either one of IgG1, IgG2, IgG3, or IgG4. The constant region sequences of human immunoglobulin gamma chain and kappa chain are described in Jay W. Ellison et al., Nucleic Acids Res. 10(13): 4071-4079, 1982 (immunoglobulin C gamma 1); Connell G E et al., Can J. Biochem. 57(6):758-767, 1979 (immunoglobulin C gamma 2); S Huck et al., Nucleic Acids Res. 14(4): 1779-1789, 1986 (immunoglobulin C gamma 3); Ellison J. W. et al., DNA, 1(1):11-18, 1981 (immunoglobulin C gamma 4) and Hieter P A et al., Cell, 22(1 Pt 1):197-207, 1980 (immunoglobulin C kappa).

Construction of Humanized Antibodies from Murine Antibody 5F4

Murine 5F4 antibody was used to make humanized antibodies by grafting its CDRs onto a human framework. To select a proper framework donor, the amino acid sequences of mouse 5F4 light and heavy chain variable regions were compared with human antibody sequences obtained from public domain. It was found that a human antibody, GenBank: AAC51710, had high homology to mouse 5F4 heavy chain variable region. And another human antibody, GenBank: AAY33352, shared high sequence homology to light chain variable region of mouse 5F4.

The humanized 5F4 light and heavy chain variable regions were assembled with synthetic oligonucleotides. The restricted enzyme sites were introduced for cloning into the constant regions containing expression plasmids. To modify the antibody affinity and efficacy, few CDR residues were randomly selected for site-directed mutagenesis. Some of them showed good efficacy compare to the un-modified version. Listed below are alignments of mouse 5F4 (m5F4) and humanized 5F4Ac.2/v17 (v17), in which the CDR regions are underlined. And the CDR modification on humanized 5F4 variants is labeled with gray shading.

| Heavy chain variable region alignment: | | | |
|---|---|---|---|
| v17 | QVQLVQSGSELKKPGASVKVSCKASGYTFTDYSMHWVRQAPGQGLEWMGWINTETGEPTY | 60 | (SEQ ID NO: 19) |
| m5F4 | QIQLVQSGPELKKPGETVKISCKASDYTFTDYSMHWVKQAPGKGLKWMGWINTETGEPTY | 60 | (SEQ ID NO: 1) |
| v17 | ADDFKGRFVFSLDTSVSTAYLQISSLKAEDTAVYYCARNKGYNLAYWGQG | 110 | |
| m5F4 | ADDFKGRFAFPLETSASTAYLQINNLKNEDTATYFCARNHGYNLAYWGQG | 110 | |
| v17 | TLVTVSS | 117 | |
| m5F4 | TLVTVSA | 117 | |
| Light chain variable region alignment: | | | |
| v17 | DIQMTQSPFSLSASVGDRVTITCKSSQNVRTAVTWYQQKPGKAPKLLIYLASNRHTGVPS | 60 | (SEQ ID NO: 20) |
| m5F4 | DIVMTQSQKFMSTSVGDRVSLTCKSSQNVRTAVTWYQQKPGQSPKALIYLASNRHTGVPD | 60 | (SEQ ID NO: 3) |
| v17 | RFSGSGSGTDFTLTISSLQPEDFATYFCLQHWYYPLTFGGGTKLEIK | 107 | |
| m5F4 | RFTGSGSGTDFTLTISNVQSEDLADYFCLQHWNYPLTFGGGTKLEIK | 107 | |

### Preparation of Chimeric and Humanized 5F4 Antibodies

Cells producing chimeric and humanized antibodies were generated by stably transfected the Flp-In CHO cells (Invitrogen, Cat. No. R758-07). More specifically, Flp-In CHO cells were transfected with appropriate plasmids by lipofectamine 2000 reagent (Invitrogen, Cat. No. 11668-027) with manufacturer's instruction. The transfectants were selected with complete medium containing 600 μg/m1 hygromycin B (Invitrogen, Cat. No. 10687-010). After 3 to 4 weeks selection and cell expansion, stable transfectants were transferred into serum free medium, and the cultured supernatants were collected. Antibodies were purified by incubation with protein G sepharose 4 Fast Flow (GE Healthcare, Cat. No. 17-0618-02) followed with manufacturer's protocol.

Chimeric and Humanized Antibodies Derived from 5F4 Induce Apoptosis of Human Plasmacytoma Cell Lines

Chimeric or humanized 5F4 antibodies were tested for its ability to induce apoptosis of human plasmacytoma cell lines in vitro. The cells (U266 or NCI-H929) were seeding in 96-well plate and incubated in the presence of purified antibodies with various concentrations at 37° C. for 6 hours. At the end of incubation, cells were either stained with Yo-Pro-1 (Invitrogen, Cat. No. Y3603) or double-stained with Annexin V-FITC and PI (Strong Biotech, Cat. No. AVK250) to measure the cell apoptosis rate. After wash steps, the cells were subjects for FACS analysis by BD FACSCalibur scanner (BD Biosciences). The results indicated that chimeric and humanized 5F4 antibodies were able to induce apoptosis of the human plasmacytoma cell lines.

Chimeric Antibody c5F4 Induces ADCC of Human Colorectal Cancer Cell

The variable regions of murine antibody 5F4 was fused with human IgG3 constant region to generate the chimeric antibody c5F4. The capability of c5F4 in inducing antibody-dependent cell-mediated cytotoxicity (ADCC) of human colorectal cancer cell line Colo205 was tested. Briefly, carboxyfluorescein succinimidyl ester (CFSE)-labeled Colo205 cells (2×10⁴ cells) were mixed with human PBMC (peripheral blood mononuclear cells) from a healthy donor at effector-to-target cell ratio of 100:1 in the presence or absence of the antibody at concentrations indicated in Table 5. After 4 hr incubation at 37° C., propidium iodide (PI) was added and the percent non-viable (PI+) cells were identified by flow cytometry. Data in Table 5 indicate that c5F4 can induce ADCC of human colorectal cancer cell.

TABLE 5
ADCC effect on human colorectal cancer cells

| | | | | hIgG | | |
|---|---|---|---|---|---|---|
| | c5F4 | | | 30 | | Target |
| | 30 ug/ml | 10 ug/ml | 3.3 ug/ml | ug/ml | No Ab | only |
| Male 1 | 40.6 | 27.4 | 17.7 | 13.1 | 13.7 | 8.8 |
| Male 2 | 47.3 | 28.5 | 13.6 | 10.0 | 12.0 | 9.1 |
| Male 3 | 36.3 | 24.6 | 19.9 | 10.6 | 13.0 | 9.1 |
| Male 4 | 25.6 | 17.4 | 17.8 | 16.8 | 13.6 | 9.1 |
| Female 1 | 21.3 | 16.7 | 15.5 | 10.6 | 13.6 | 8.8 |
| Female 2 | 36.7 | 19.7 | 13.1 | 11.8 | 12.7 | 10.6 |
| Female 3 | 41.7 | 27.6 | 16.1 | 11.5 | 12.7 | 10.6 |
| Female 4 | 35.5 | 21.8 | 17.2 | 13.4 | 12.5 | 10.6 |

Numbers indicate % PI+ cells among CFSE+ (target) cells.

### Suppression of Multiple Myeloma Tumor Growth in SCID by a Humanized 5F4 Antibody

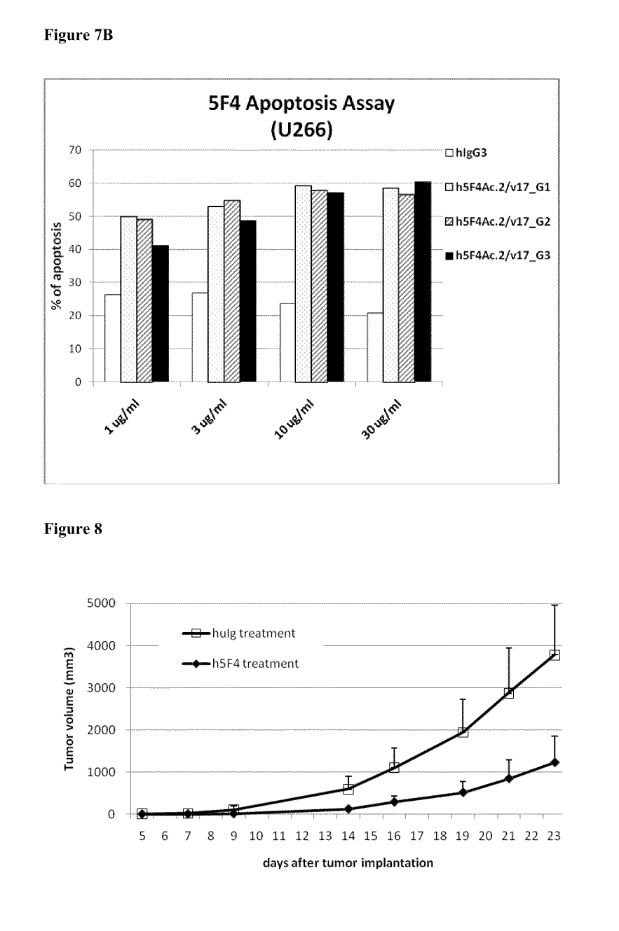

Therapeutic effect of the anti-ORP150 antibody was further explored in a multiple myeloma xenograft model in C.B17-SCID mice. Mice were implanted subcutaneously with 1×10⁷ L363 cells into the hind flank, and treated intraperitoneally with humanized 5F4Ac.2/v17 (human IgG3, kappa) or a control human IgG (huIg) at 20 mg/kg three times weekly. Tumor volume was determined by caliper measurement and calculated using the formula (length×width²)/2. Experiment was terminated 3 weeks after tumor inoculation, on which day mice had received 10 injections of antibodies. As shown in FIG. 8, treatment with h5F4 gives a significant suppressive effect on tumor growth, while no anti-tumor activity was observed in mice treated with control huIg (on day 23, p=0.015). These results indicate the anti-tumor activity of anti-ORP150 antibody for multiple myeloma therapy.

Although the foregoing invention has been described in some detail by way of illustration and example for purposes of clarity of understanding, the descriptions and examples should not be construed as limiting the scope of the invention.

### REFERENCES

Mentzer, S. J., Remold-O'Donnell, E., Crimmins, M. A., Bierer, B. E., Rosen, F. S., and Burakoff, S. J. (1987) Sialophorin, a surface sialoglycoprotein defective in the Wiskott-Aldrich syndrome, is involved in human T lymphocyte proliferation. J. Exp. Med. 165(5):1383-92.
Pallant, A., Eskenazi, A., Mattei, M G., Fournier, R. E. K., Carlsson, S. R., Fukuda, M., and Frelinger, J. G. (1989) Characterization of cDNA encoding human leukosialin and localization of the leukosialin gene to chromosome 16. Proc. Natl. Acad. Sci. USA 86:1328-32.
Ozawa et al., 150-kDa oxygen-regulated protein (ORP150) suppresses hypoxia-induced apoptotic cell death. J. Biol. Chem. 1999; 274(10):6397-404.
Tamatani et al., ORP150 protects against hypoxia/ischemia-induced neuronal death. Nat Med. 2001; 7(3):317-23.
Ozawa et al., Regulation of tumor angiogenesis by oxygen-regulated protein 150, an inducible endoplasmic reticulum chaperone. Cancer Res. 2001; 61(10):4206-13.
Miyagi et al., Antitumor effect of reduction of 150-kDa oxygen-regulated protein expression on human prostate cancer cells. Int. J. Urol. 2002; 9(10):577-85.
Asahi et al., Immunohistochemical detection of the 150-kDa oxygen-regulated protein in bladder cancer. BJU Int. 2002; 90(4):462-6.
Stojadinovic et al., HYOU1/Orp150 expression in breast cancer. Med. Sci. Monit. 2007; 13(11):BR231-239.
Multhoff et al. (2007) Therapeutic and Diagnostic Anti-Hsp70 Antibodies. United States Patent 2007/0231337.

What is claimed is:

1. An isolated antibody that specifically binds to an ORP150 polypeptide expressed on the cell surface of a plasmacytoma cell, a multiple myeloma cell, a colorectal cancer cell, a gastric cancer cell, or an esophageal cancer cell.

2. The antibody of claim 1, wherein the antibody induces apoptosis of the plasmacytoma cell, the multiple myeloma cell, or the gastric cancer cell after binding to the cell surface of the plasmacytoma cell, the multiple myeloma cell, or the gastric cancer cell in the absence of cytotoxin conjugation or immune effector function.

3. The antibody of claim 1, wherein the antibody induces complement-dependent cytotoxicity in the cell after binding to the cell surface of the cell.

4. The antibody of claim 1, wherein the antibody induces antibody-dependent cell-mediated cytotoxicity in the cell after binding to the cell surface of the cell.

5. The antibody of claim 1, wherein the antibody interacts with amino acids within residues 673-752 of SEQ ID NO:17.

6. The antibody of claim 1, wherein the antibody interacts with amino acids within residues 723-732 of SEQ ID NO:17.

7. The antibody of claim 1, wherein the antibody interacts with amino acids within residues 701-800 of SEQ ID NO:17.

8. The antibody of claim 1, wherein the antibody binds to a polypeptide comprising amino acids 673-752, 723-732, or 701-800 of SEQ ID NO:17.

9. The antibody of claim 1, wherein the antibody is a monoclonal antibody.

10. The antibody of claim 1, wherein the antibody is a human antibody.

11. The antibody of claim 1, wherein the antibody is a chimeric antibody.

12. The antibody of claim 1, wherein the antibody is a humanized antibody.

13. The antibody of claim 1, wherein the antibody is a bispecific antibody.

14. The antibody of claim 1, wherein the antibody is conjugated to a cytotoxin.

15. The antibody of claim 1, wherein the antibody comprises a heavy chain variable region comprising the three complementary determining regions of SEQ ID NO:1 and/or a light chain variable region comprising the three complementary determining regions of SEQ ID NO:3.

16. The antibody of claim 1, wherein the antibody comprises a heavy chain variable region comprising the amino acid sequence of SEQ ID NO:1 and/or a light chain variable region comprising the amino acid sequence of SEQ ID NO:3.

17. The antibody of claim 1, wherein the antibody comprises a heavy chain variable region comprising the three complementary determining regions of SEQ ID NO:5 and/or a light chain variable region comprising the three complementary determining regions of SEQ ID NO:7.

18. The antibody of claim 1, wherein the antibody comprises a heavy chain variable region comprising the amino acid sequence of SEQ ID NO:5 and/or a light chain variable region comprising the amino acid sequence of SEQ ID NO:7.

19. The antibody of claim 1, wherein the antibody comprises a heavy chain variable region comprising the three complementary determining regions of SEQ ID NO:9 and/or a light chain variable region comprising the three complementary determining regions of SEQ ID NO:11.

20. The antibody of claim 1, wherein the antibody comprises a heavy chain variable region comprising the amino acid sequence of SEQ ID NO:9 and/or a light chain variable region comprising the amino acid sequence of SEQ ID NO:11.

21. The antibody of claim 1, wherein the antibody comprises a heavy chain variable region comprising the three complementary determining regions of SEQ ID NO:13 and/or a light chain variable region comprising the three complementary determining regions of SEQ ID NO:15.

22. The antibody of claim 1, wherein the antibody comprises a heavy chain variable region comprising the amino acid sequence of SEQ ID NO:13 and/or a light chain variable region comprising the amino acid sequence of SEQ ID NO:15.

23. The antibody of claim 1, wherein the antibody comprises a heavy chain variable region comprising the three complementary determining regions of SEQ ID NO:19 and a light chain variable region comprising the three complementary determining regions of SEQ ID NO:20.

24. The antibody of claim 1, wherein the antibody comprises a heavy chain variable region comprising the amino acid sequence of SEQ ID NO:19 and/or a light chain variable region comprising the amino acid sequence of SEQ ID NO:20.

25. A polynucleotide comprising a nucleic acid sequence encoding an antibody of claim 1.

26. A vector comprising a nucleic acid sequence encoding an antibody of claim 1.

27. A cell comprising a vector of claim 26.

28. A cell that produces an antibody of claim 1.

29. A composition comprising the antibody of claim 1, and a pharmaceutically acceptable carrier.

30. An anti-idiotypic antibody that specifically binds to the antibody of claim 1.

31. The anti-idiotypic antibody of claim 30, wherein the anti-idiotypic antibody is a monoclonal antibody.

32. The anti-idiotypic antibody of claim 30, wherein the anti-idiotypic antibody is a human antibody.

33. The anti-idiotypic antibody of claim 30, wherein the anti-idiotypic antibody is a chimeric antibody.

34. The anti-idiotypic antibody of claim 30, wherein the anti-idiotypic antibody is a humanized antibody.

35. A polynucleotide comprising a nucleic acid sequence encoding an anti-idiotypic antibody of claim 30.

36. A vector comprising a nucleic acid sequence encoding an anti-idiotypic antibody of claim 30.

37. A cell comprising a vector of claim 36.

38. A cell that produces an anti-idiotypic antibody of claim 30.

39. A composition comprising an anti-idiotypic antibody of claim 30, and a pharmaceutically acceptable carrier.

40. The composition of claim 39, further comprising a polypeptide comprising an extracellular domain or fragment thereof of an ORP150 polypeptide expressed on the cell surface of a plasmacytoma cell, a multiple myeloma cell, a colorectal cancer cell, a gastric cancer cell, or an esophageal cancer cell.

41. The composition of claim 40, wherein the polypeptide comprises amino acids 723-732, 673-752, or 701-800 of SEQ ID NO:17.

42. A composition comprising a polypeptide comprising an extracellular domain or fragment thereof of an ORP150 polypeptide expressed on the cell surface of a plasmacytoma cell, a multiple myeloma cell, a colorectal cancer cell, a gastric cancer cell, or an esophageal cancer cell, and a pharmaceutically acceptable carrier.

43. The composition of claim 42, further comprising an adjuvant.

44. The composition of claim 42, wherein the polypeptide comprises amino acids 723-732, 673-752, or 701-800 of SEQ ID NO:17.

45. A method for treating cancer in an individual having cancer selected from the group consisting of plasmacytoma, multiple myeloma, colorectal cancer, gastric cancer, and esophageal cancer, comprising administering to the individual an effective amount of one or more antibodies of claim 1.

46. The method of claim 45, wherein the antibody is conjugated to a cytotoxin.

47. A method for treating cancer in an individual having cancer selected from the group consisting of plasmacytoma, multiple myeloma, colorectal cancer, gastric cancer, and esophageal cancer, comprising administering to the individual (i) one or more antibodies of claim 1 and (ii) another anti-cancer agent, whereby the antibody and the anti-cancer agent in conjunction provides effective treatment of the cancer in the individual.

48. The method of claim 47, wherein the antibody is conjugated to a cytotoxin.

49. The method of claim 47, wherein the anti-cancer agent is a chemotherapeutic agent.

50. A method for delaying the development of cancer in an individual having cancer selected from the group consisting of plasmacytoma, multiple myeloma, colorectal cancer, gastric cancer, and esophageal cancer or having an increased risk for cancer selected from the group consisting of plasmacytoma, multiple myeloma, colorectal cancer, gastric cancer, and esophageal cancer, comprising administering to the individual an effective amount of one or more antibodies of claim 1.

51. The method of claim 50, wherein the antibody is conjugated to a cytotoxin.

52. A method for treating cancer in an individual having cancer selected from the group consisting of plasmacytoma, multiple myeloma, colorectal cancer, gastric cancer, and esophageal cancer comprising administering to the individual an effective amount of one or more anti-idiotypic antibodies of claim 30.

53. The method of claim 52, further comprising administering to the individual a polypeptide comprising an extracellular domain or fragment thereof of an ORP150 polypeptide expressed on the cell surface of a plasmacytoma cell, a multiple myeloma cell, a colorectal cancer cell, a gastric cancer cell, or an esophageal cancer cell.

54. A method for preventing or delaying the development of cancer in an individual having cancer selected from the group consisting of plasmacytoma, multiple myeloma, colorectal cancer, gastric cancer, and esophageal cancer or having an increased risk for cancer selected from the group consisting of plasmacytoma, multiple myeloma, colorectal cancer, gastric cancer, and esophageal cancer, comprising administering to the individual an effective amount of one or more anti-idiotypic antibodies of claim 30.

55. The method of claim 54, further comprising administering to the individual an extracellular domain or fragment thereof of an ORP150 polypeptide expressed on the cell surface of a plasmacytoma cell, a multiple myeloma cell, a colorectal cancer cell, a gastric cancer cell, or an esophageal cancer cell.

56. A method for treating cancer in an individual having cancer selected from the group consisting of plasmacytoma, multiple myeloma, colorectal cancer, gastric cancer, and esophageal cancer, comprising administering to the individual an effective amount of a polypeptide comprising an extracellular domain or fragment thereof of an ORP150 polypeptide expressed on the cell surface of a plasmacytoma cell, a multiple myeloma cell, a colorectal cancer cell, a gastric cancer cell, or an esophageal cancer cell.

57. A method for preventing or delaying the development of cancer in an individual having cancer selected from the group consisting of plasmacytoma, multiple myeloma, colorectal cancer, gastric cancer, and esophageal cancer or having an increased risk for cancer selected from the group consisting of plasmacytoma, multiple myeloma, colorectal cancer, gastric cancer, and esophageal cancer, comprising administering to the individual an effective amount of a polypeptide comprising an extracellular domain or fragment thereof of an ORP150 polypeptide expressed on the cell surface of a plasmacytoma cell, a multiple myeloma cell, a colorectal cancer cell, a gastric cancer cell, or an esophageal cancer cell.

58. A method for diagnosing plasmacytoma or multiple myeloma or an increased risk for plasmacytoma or multiple myeloma in an individual comprising contacting a bone marrow sample from the individual with of one or more antibodies of claim 1, whereby the ability of one or more of the antibodies to bind to the cell surface of a bone marrow cell indicates that the individual has plasmacytoma or multiple myeloma or has an increased risk for plasmacytoma or multiple myeloma.

59. A method for diagnosing colorectal cancer or an increased risk for colorectal cancer in an individual comprising contacting a sample comprising a colon or rectum cell from the individual with of one or more antibodies of claim 1, whereby the ability of one or more of the antibodies to bind to the cell surface of the colon or rectum cell indicates that the individual has colorectal cancer or has an increased risk for colorectal cancer.

60. A method for diagnosing gastric cancer or an increased risk for gastric cancer in an individual comprising contacting a sample comprising a gastric cell from the individual with of one or more antibodies of claim 1, whereby the ability of one or more of the antibodies to bind to the cell surface of the gastric cell indicates that the individual has gastric cancer or has an increased risk for gastric cancer.

61. A method for diagnosing esophageal cancer or an increased risk for esophageal cancer in an individual comprising contacting a sample comprising an esophageal cell from the individual with of one or more antibodies of claim 1, whereby the ability of one or more of the antibodies to bind to the cell surface of the esophageal cell indicates that the individual has esophageal cancer or has an increased risk for esophageal cancer.

62. A method of selecting a therapy for an individual having plasmacytoma or multiple myeloma comprising contacting a bone marrow sample from the individual with of one or more antibodies of claim 1, whereby the ability of one or more of the antibodies to bind to the cell surface of a bone marrow cell indicates that the one or more antibodies are useful for treating the plasmacytoma or multiple myeloma in the individual.

63. A method of selecting a therapy for an individual having colorectal cancer in an individual comprising contacting a sample comprising a colon or rectum cell from the individual with of one or more antibodies of claim 1, whereby the ability of one or more of the antibodies to bind to the cell surface of the colon or rectum cell indicates that the one or more antibodies are useful for treating the colorectal cancer in the individual.

64. A method of selecting a therapy for an individual having gastric cancer in an individual comprising contacting a sample comprising a gastric cell from the individual with of one or more antibodies of claim 1, whereby the ability of one or more of the antibodies to bind to the cell surface of the gastric cell indicates that the one or more antibodies are useful for treating the gastric cancer in the individual.

65. A method of selecting a therapy for an individual having esophageal cancer in an individual comprising contacting a sample comprising an esophageal cell from the individual with of one or more antibodies of claim 1, whereby the ability of one or more of the antibodies to bind to the cell surface of the esophageal cell indicates that the one or more antibodies are useful for treating the esophageal cancer in the individual.

66. A kit comprising a pharmaceutical composition comprising an antibody of claim 1, and instructions for administering an effective amount of the pharmaceutical composition to an individual for treating cancer selected from the group consisting of plasmacytoma, multiple myeloma, colorectal cancer, gastric cancer, and esophageal cancer.

67. A kit comprising a pharmaceutical composition comprising an antibody of claim 1, and instructions for administering an effective amount of the pharmaceutical composition to an individual for delaying the development of cancer selected from the group consisting of plasmacytoma, multiple myeloma, colorectal cancer, gastric cancer, and esophageal cancer.

68. A kit comprising a pharmaceutical composition comprising an anti-idiotypic antibody of claim 30, and instructions for administering an effective amount of the pharmaceutical composition to an individual for treating cancer selected from the group consisting of plasmacytoma, multiple myeloma, colorectal cancer, gastric cancer, and esophageal cancer.

69. A kit comprising a pharmaceutical composition comprising an anti-idiotypic antibody of claim 30, and instructions for administering an effective amount of the pharmaceutical composition to an individual for delaying the development of cancer selected from the group consisting of plasmacytoma, multiple myeloma, colorectal cancer, gastric cancer, and esophageal cancer.

70. A kit comprising a pharmaceutical composition comprising a polypeptide comprising an extracellular domain or fragment thereof of an ORP150 polypeptide expressed on the cell surface of a plasmacytoma cell, a multiple myeloma cell, a colorectal cancer cell, a gastric cancer cell, or an esophageal cancer cell, and instructions for administering an effective amount of the pharmaceutical composition to an individual for treating cancer selected from the group consisting of plasmacytoma, multiple myeloma, colorectal cancer, gastric cancer, and esophageal cancer.

71. A kit comprising a pharmaceutical composition comprising a polypeptide comprising an extracellular domain or fragment thereof of an ORP150 polypeptide expressed on the cell surface of a plasmacytoma cell, a multiple myeloma cell, a colorectal cancer cell, a gastric cancer cell, or an esophageal cancer cell, and instructions for administering an effective amount of the pharmaceutical composition to an individual for preventing or delaying the development of cancer selected from the group consisting of plasmacytoma, multiple myeloma, colorectal cancer, gastric cancer, and esophageal cancer.

72. A kit comprising an antibody of claim 1, and instructions for using the antibody to diagnose cancer selected from the group consisting of plasmacytoma, multiple myeloma, colorectal cancer, gastric cancer, and esophageal cancer.

73. A method of producing an antibody of claim 1, comprising administering a polypeptide comprising an ORP150 polypeptide or fragment thereof to an animal, and selecting an antibody produced by the animal that specifically binds to an ORP150 polypeptide expressed on the cell surface of a plasmacytoma cell, a multiple myeloma cell, a colorectal cancer cell, a gastric cancer cell, or an esophageal cancer cell.

74. The method of claim 73, wherein the antibody is isolated.

75. The method of claim 73, wherein part or all of a cell membrane comprising an ORP150 polypeptide or fragment thereof is administered to the animal.

76. The method of claim 73, wherein the polypeptide comprises amino acids 723-732, 673-752, or 701-800 of SEQ ID NO:17.

77. The method of claim 73, wherein the animal is a mammal.

78. A method of producing an anti-idiotypic antibody, comprising administering an antibody of claim 1 or fragment thereof to an animal, and selecting an anti-idiotypic antibody produced by the animal that specifically binds to the antibody of claim 1 or fragment thereof.

79. The method of claim 78, wherein the anti-idiotypic antibody is isolated.

80. The method of claim 78, wherein the animal is a mammal.
